(19) United States
(12) Patent Application Publication
(10) Pub. No.: US 2026/0240208 A1
(43) Pub. Date: Aug. 20, 2026

(54) FLAVANONE COMPOUNDS AND THEIR USE AS FLAVOR MODIFIERS

(71) Applicant: Firmenich Incorporated, Plainsboro, NJ (US)

(72) Inventors: Joseph R. Fotsing, San Diego, CA (US); Sergiy Levin, San Diego, CA (US)

(21) Appl. No.: 19/159,288

(22) PCT Filed: Feb. 16, 2024
(86) PCT No.: PCT/US2024/016158

§ 371 Date: Aug. 22, 2025

Related U.S. Application Data
(60) Provisional application No. 63/448,261, filed on Feb. 25, 2023.

(51) Int. Cl. A23L 27/20; A23L 27/00; A23L 27/30; C07D 311/30

(52) U.S. Cl. CPC A23L 27/2052; A23L 27/204; A23L 27/30; A23L 27/86; A23L 27/88; C07D 311/30

(57) ABSTRACT

The present disclosure generally relates to 3-acyloxyflavanone compounds and the use of such compounds to impart and/or enhance sweetness, mask bitterness, enhance mouthfeel, or mask astringency. In certain embodiments, such compounds are used in combination with other flavonoids or dihydrochalcones. In certain aspects, the disclosure provides ingestible compositions that include such flavanone compounds. In some related aspects, the ingestible compositions are, or are included within, various flavored products, such as food products, beverage products, pharmaceutical products, or oral care products.

### TECHNICAL FIELD

The present disclosure generally relates to 3-acyloxyflavanone compounds and the use of such compounds to impart and/or enhance sweetness, mask bitterness, enhance mouthfeel, or mask astringency. In certain embodiments, such compounds are used in combination with other flavonoids or dihydrochalcones. In certain aspects, the disclosure provides ingestible compositions that include such flavanone compounds. In some related aspects, the ingestible compositions are, or are included within, various flavored products, such as food products, beverage products, pharmaceutical products, or oral care products.

### DESCRIPTION OF RELATED ART

The taste system provides sensory information about the chemical composition of the external world. Taste transduction is one of the more sophisticated forms of chemically triggered sensation in animals. Signaling of taste is found throughout the animal kingdom, from simple metazoans to the most complex of vertebrates. Mammals are believed to have five basic taste modalities: sweet, bitter, sour, salty, and umami.

Sweetness is the taste most commonly perceived when eating foods rich in sugars. Mammals generally perceive sweetness to be a pleasurable sensation, except in excess. Caloric sweeteners, such as sucrose and fructose, are the prototypical examples of sweet substances. Although a variety of no-calorie and low-calorie substitutes exist, these caloric sweeteners are still the predominant means by which comestible products induce the perception of sweetness upon consumption.

Metabolic disorders and related conditions, such as obesity, diabetes, and cardiovascular disease, are major public health concerns throughout the world. And their prevalence is increasing at alarming rates in almost every developed country. Caloric sweeteners are a key contributor to this trend, as they are included in various packaged food and beverage products to make them more palatable to consumers. In many cases, no-calorie or low-calorie substitutes can be used in foods and beverages in place of sucrose or fructose. Even so, these compounds impart sweetness differently from caloric sweeteners, and a number of consumers fail to view them as suitable alternatives. Moreover, such compounds may be difficult to incorporate into certain products. In some instances, they may be used as partial replacements for caloric sweeteners, but their mere presence can cause many consumers to perceive unpleasant off-tastes including, astringency, bitterness, and metallic and licorice tastes. Thus, lower-calorie sweeteners face certain challenges to their adoption.

Sweetness enhancement provides an alternative approach to overcoming some of adoption challenges faced by lower-calorie sweeteners. Such compounds can be used in combination with sucrose or fructose to enhance their sweetness, thereby permitting the use of lower quantities of such caloric sweeteners in various food or beverage products. But, in addition to enhancing the perceived sweetness of the primary sweetener, such compounds nevertheless alter the perceived taste of the sweetener. Thus, many consumers find that it is less pleasurable to consume such sweetness-enhanced products in comparison to unenhanced alternatives having higher calories. Thus, there is a continuing need to discover compounds that enhance the sweetness of caloric sweeteners without altering their perceived taste in a way that detracts from the pleasure that consumers experience in eating or drinking products containing such sweeteners.

### SUMMARY

The present disclosure relates to the discovery that certain flavanones and related compounds impart and/or enhance sweetness in a more natural-tasting way, and, in some cases, can also be used to mask bitterness, enhance mouthfeel, or mask astringency.

In a first aspect, the disclosure provides a flavor-modifying compound, which is a compound of formula (I):

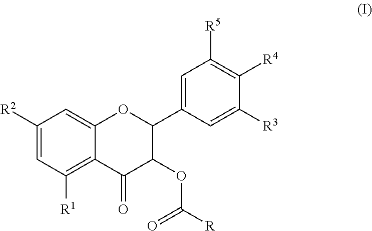

or a salt thereof, wherein:
R¹ and R² are, independently of each other, a hydrogen atom, —OH, or —OCH₃;
R³, R⁴, and R⁵ are, independently of each other, a hydrogen atom, —OH, or —OCH₃; and
R is methyl, ethyl, isopropyl, or benzyl.

In a second aspect, the disclosure provides uses of the flavor-modifying compounds of the first aspect to enhance a sweet taste of an ingestible composition. In certain related aspects, the disclosure provides methods of enhancing a sweet taste of an ingestible composition, the method comprising introducing a flavor-modifying compound of the first aspect to an ingestible composition. In some embodiments, the ingestible composition comprises one or more sweeteners, such as caloric or non-caloric sweeteners. In some embodiments, the ingestible compositions comprise one or more flavonoids or dihydrochalcones.

In a third aspect, the disclosure provides uses of the flavor-modifying compounds of the first aspect to reduce a bitter taste of an ingestible composition. In certain related aspects, the disclosure provides methods of reducing a bitter taste of an ingestible composition, the method comprising introducing a flavor-modifying compound of the first aspect to an ingestible composition. In some embodiments, the ingestible composition comprises one or more bitter tastants, such as certain high-intensity sweeteners, caffeine, tannins, pharmaceutical APIs, and the like. In some embodiments, the ingestible compositions comprise one or more flavonoids or dihydrochalcones.

In a fourth aspect, the disclosure provides uses of the flavor-modifying compounds of the first aspect to reduce an astringent taste of an ingestible composition. In certain related aspects, the disclosure provides methods of reducing an astringent taste of an ingestible composition, the method comprising introducing a flavor-modifying compound of the first aspect to an ingestible composition. In some embodiments, the ingestible composition comprises one or more astringent compounds. Non-limiting examples of such uses and methods include reducing the lingering licorice aftertaste of certain high-intensity sweeteners, such as stevia-based high-intensity sweeteners. In some embodiments, the ingestible compositions comprise one or more flavonoids or dihydrochalcones.

In a fifth aspect, the disclosure provides uses of the flavor-modifying compounds of the first aspect to enhance mouthfeel of an ingestible composition. In certain related aspects, the disclosure provides methods of enhancing mouthfeel of an ingestible composition, the method comprising introducing a flavor-modifying compound of the first aspect to an ingestible composition. In some embodiments, the ingestible compositions comprise one or more flavonoids or dihydrochalcones.

In a sixth aspect, the disclosure provides an ingestible composition comprising one or more flavor-modifying compounds of the first aspect. In some embodiments, the ingestible composition comprises one or more sweeteners. In some embodiments, the ingestible composition comprises one or more bitter tastants, such as a high-intensity sweetener or certain bitter compounds.

In a seventh aspect, the disclosure provides a flavored product, which comprises an ingestible composition of the sixth aspect. In some embodiments, the flavored product is a food or beverage product. In some embodiments, the flavored product is an oral care product or a pharmaceutical product.

Further aspects, and embodiments thereof, are set forth below in the Detailed Description, the Drawings, the Abstract, and the Claims.

### BRIEF DESCRIPTION OF THE DRAWINGS

The following drawings are provided for purposes of illustrating various embodiments of the compositions and methods disclosed herein. The drawings are provided for illustrative purposes only, and are not intended to describe any preferred compositions or preferred methods, or to serve as a source of any limitations on the scope of the claimed inventions.

FIG. 1 shows a chemical formula that represents the flavor-modifying compounds disclosed herein, wherein R¹ and R² are, independently of each other, a hydrogen atom, —OH, or —OCH₃; R³, R⁴, and R⁵ are, independently of each other, a hydrogen atom, —OH, or —OCH₃; and R is methyl, ethyl, isopropyl, or benzyl.

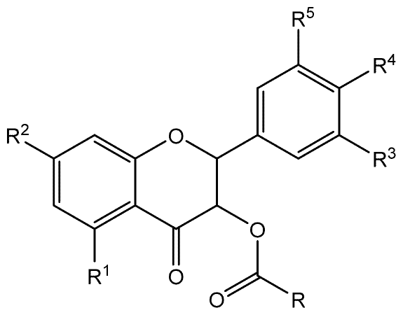

### DETAILED DESCRIPTION

The following Detailed Description sets forth various aspects and embodiments provided herein. The description is to be read from the perspective of the person of ordinary skill in the relevant art. Therefore, information that is well known to such ordinarily skilled artisans is not necessarily included.

### Definitions

The following terms and phrases have the meanings indicated below, unless otherwise provided herein. This disclosure may employ other terms and phrases not expressly defined herein. Such other terms and phrases have the meanings that they would possess within the context of this disclosure to those of ordinary skill in the art. In some instances, a term or phrase may be defined in the singular or plural. In such instances, it is understood that any term in the singular may include its plural counterpart and vice versa, unless expressly indicated to the contrary.

A “sweetener” refers to a compound or ingestibly acceptable salt thereof that elicits a detectable sweet taste in a subject, e.g., a compound that activates the TIR2 and TIR3 taste receptors in vivo or in vitro.

A “bitter tastant” refers to a compound or ingestibly acceptable salt thereof that elicits a detectable bitter taste in a subject, e.g., a compound that activates one or more T2R taste receptors in vivo or in vitro.

As used herein, the singular forms “a,” “an,” and “the” include plural referents unless the context clearly dictates otherwise. For example, reference to “a substituent” encompasses a single substituent as well as two or more substituents, and the like.

As used herein, “for example,” “for instance,” “such as,” or “including” are meant to introduce examples that further clarify more general subject matter. Unless otherwise expressly indicated, such examples are provided only as an aid for understanding embodiments illustrated in the present disclosure, and are not meant to be limiting in any fashion. Nor do these phrases indicate any kind of preference for the disclosed embodiment.

As used herein, “comprise” or “comprises” or “comprising” or “comprised of” refer to groups that are open, meaning that the group can include additional members in addition to those expressly recited. For example, the phrase, “comprises A” means that A must be present, but that other members can be present too. The terms “include,” “have,” and “composed of” and their grammatical variants have the same meaning. In contrast, “consist of” or “consists of” or “consisting of” refer to groups that are closed. For example, the phrase “consists of A” means that A and only A is present.

As used herein, “optionally” means that the subsequently described event(s) may or may not occur. In some embodiments, the optional event does not occur. In some other embodiments, the optional event does occur one or more times.

As used herein, “or” is to be given its broadest reasonable interpretation, and is not to be limited to an either/or construction. Thus, the phrase “comprising A or B” means that A can be present and not B, or that B is present and not A, or that A and B are both present. Further, if A, for example, defines a class that can have multiple members, e.g., A₁ and A₂, then one or more members of the class can be present concurrently.

Chemical structures are often shown using the “skeletal” format, such that carbon atoms are not explicitly shown, and hydrogen atoms attached to carbon atoms are omitted entirely. For example, the structure

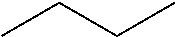

represents butane (i.e., n-butane). Furthermore, aromatic groups, such as benzene, are represented by showing one of the contributing resonance structures. For example, the structure

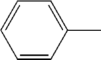

represents toluene.

As used herein, the term “flavor-modifying compound” refers to any compound of formula (I), or salts thereof, and any embodiments thereof set forth herein.

Other terms are defined in other portions of this description, even though not included in this subsection.

### Flavor-Modifying Compounds

In certain aspects, the disclosure provides a flavor-modifying compound, which is a compound of formula (I):

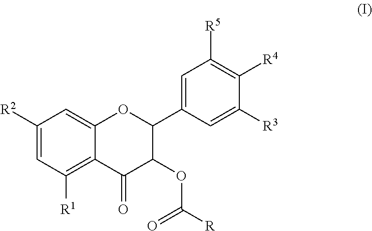

or a salt thereof, wherein:
R¹ and R² are, independently of each other, a hydrogen atom, —OH, or —OCH₃;
R³, R⁴, and R⁵ are, independently of each other, a hydrogen atom, —OH, or —OCH₃; and
R is methyl, ethyl, isopropyl, or benzyl.

The variables R¹ and R² can have any suitable value within the scope of the definitions set forth above. In some embodiments, at least one of R¹ and R² is not a hydrogen atom. In some embodiments, R¹ and R² are, independently of each other, a hydrogen atom or —OH. In some embodiments, R¹ is —OH and R² is a hydrogen atom. In some embodiments, R¹ is a hydrogen atom and R² is —OH. In some embodiments, R¹ is —OH and R² is —OH.

The variables R³, R⁴, and R⁵ can have any suitable value within the scope of the definitions set forth above. In some embodiments, one of R³ and R⁵ is a hydrogen atom. In some embodiments, both of R³ and R⁵ are each a hydrogen atom. In some embodiments, R⁴ is-OH and R³ and R⁵ are each a hydrogen atom. In some embodiments, R³ is —OH, R⁴ is —OCH₃, and R⁵ is a hydrogen atom.

The variable R can have any suitable value within the scope of the definitions set forth above. In some embodiments, R is methyl or ethyl. In some embodiments, R is methyl. In some embodiments, R is ethyl. In some embodiments, R is isopropyl. In some embodiments, R is benzyl.

Table 1 provides examples of flavor-modifying compounds of the present disclosure. In some embodiments, the flavor-modifying compound is Compound 101 or a comestibly acceptable salt thereof. In some embodiments, the flavor-modifying compound is Compound 102 or a comestibly acceptable salt thereof. In some embodiments, the flavor-modifying compound is Compound 103 or a comestibly acceptable salt thereof. In some embodiments, the flavor-modifying compound is Compound 104 or a comestibly acceptable salt thereof. In some embodiments, the flavor-modifying compound is Compound 105 or a comestibly acceptable salt thereof. In some embodiments, the flavor-modifying compound is Compound 106 or a comestibly acceptable salt thereof.

TABLE 1

| No. | Compound |
|---|---|
| 101 | 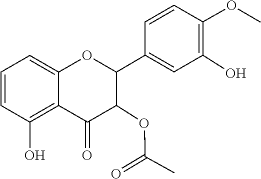 [SMILES: COc1ccc(C2Oc3cccc(O)c3C(=O)C2OC(C)=O)cc1O] |
| | 5-hydroxy-2-(3-hydroxy-4-methoxy- |
| | phenyl)-4-oxochroman-3-yl acetate |
| 102 | 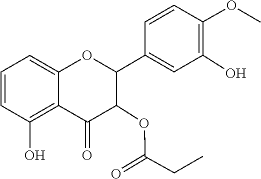 [SMILES: CCC(=O)OC1C(=O)c2c(O)cccc2OC1c1ccc(OC)c(O)c1] |
| | 5-hydroxy-2-(3-hydroxy-4-methoxy- |
| | phenyl)-4-oxochroman-3-yl propionate |
| 103 | 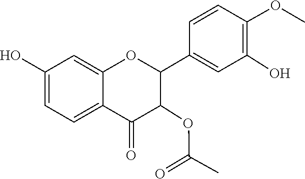 [SMILES: COc1ccc(C2Oc3cc(O)ccc3C(=O)C2OC(C)=O)cc1O] |
| | 7-hydroxy-2-(3-hydroxy-4-methoxyphenyl)- |
| | 4-oxochroman-3-yl acetate |
| 104 | 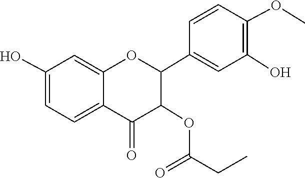 [SMILES: CCC(=O)OC1C(=O)c2ccc(O)cc2OC1c1ccc(OC)c(O)c1] |
| | 7-hydroxy-2-(3-hydroxy-4-methoxyphenyl)- |
| | 4-oxochroman-3-yl propionate |
| 105 | 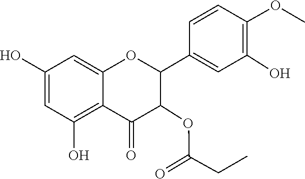 [SMILES: CCC(=O)OC1C(=O)c2c(O)cc(O)cc2OC1c1ccc(OC)c(O)c1] |
| | 5,7-dihydroxy-2-(3-hydroxy-4-methoxyphenyl)- |
| | 4-oxochroman-3-yl propionate |
| 106 | 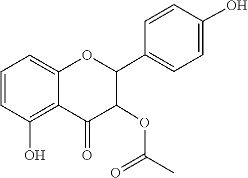 [SMILES: CC(=O)OC1C(=O)c2c(O)cccc2OC1c1ccc(O)cc1] |
| | 5-hydroxy-2-(4-hydroxyphenyl)-4- |
| | oxochroman-3-yl acetate |

Where the flavor-modifying compounds disclosed herein have at least one chiral center, they may exist as individual enantiomers and diastereomers or as mixtures of such isomers. In some embodiments in connection with the second aspect, the sweet-enhancing compound has substantial enantiomeric purity.

Separation of the individual isomers or selective synthesis of the individual isomers is accomplished by application of various methods which are well known to practitioners in the art. Unless otherwise indicated (e.g., where the stereochemistry of a chiral center is explicitly shown), all such isomers and mixtures thereof are included in the scope of the compounds disclosed herein. Furthermore, compounds disclosed herein may exist in one or more crystalline or amorphous forms. Unless otherwise indicated, all such forms are included in the scope of the compounds disclosed herein including any polymorphic forms. In addition, some of the compounds disclosed herein may form solvates with water (i.e., hydrates) or common organic solvents. Unless otherwise indicated, such solvates are included in the scope of the compounds disclosed herein.

The skilled artisan will recognize that some structures described herein may be resonance forms or tautomers of compounds that may be fairly represented by other chemical structures, even when kinetically; the artisan recognizes that such structures may only represent a very small portion of a sample of such compound(s). Such compounds are considered within the scope of the structures depicted, though such resonance forms or tautomers are not represented herein.

Isotopes may be present in the compounds described. Each chemical element as represented in a compound structure may include any isotope of said element. For example, in a compound structure a hydrogen atom may be explicitly disclosed or understood to be present in the compound. At any position of the compound that a hydrogen atom may be present, the hydrogen atom can be any isotope of hydrogen, including but not limited to hydrogen-1 (protium) and hydrogen-2 (deuterium). Thus, reference herein to a compound encompasses all potential isotopic forms unless the context clearly dictates otherwise.

In some embodiments, the flavor-modifying compounds disclosed herein are capable of forming acid and/or base salts by virtue of the presence of phenol, amino, and/or carboxyl groups or groups similar thereto. Comestibly acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Comestibly acceptable salts can be formed using inorganic and organic bases. Inorganic bases from which salts can be derived include, for example, bases that contain sodium, potassium, lithium, ammonium, calcium, magnesium, iron, zinc, copper, manganese, aluminum, and the like; particularly preferred are the ammonium, potassium, sodium, calcium and magnesium salts. In some embodiments, treatment of the compounds disclosed herein with an inorganic base results in loss of a labile hydrogen from the compound to afford the salt form including an inorganic cation such as Li⁺, Na⁺, K⁺, Mg²⁺ and Ca²⁺ and the like. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine. In some embodiments, the salts are comestibly acceptable salts, which are salts suitable for inclusion in ingestible compositions, such as food or beverage products. In some embodiments, the comestibly acceptable salts are sodium or potassium salts.

### Uses and Methods

In certain aspects, the disclosure provides uses of the flavor-modifying compounds of the first aspect to enhance a sweet taste of an ingestible composition. In certain related aspects, the disclosure provides methods of enhancing a sweet taste of an ingestible composition, the method comprising introducing a flavor-modifying compound of the first aspect to an ingestible composition.

In certain aspects, the disclosure provides uses of the flavor-modifying compounds of the first aspect to reduce a bitter taste of an ingestible composition. In certain related aspects, the disclosure provides methods of reducing a bitter taste of an ingestible composition, the method comprising introducing a flavor-modifying compound of the first aspect to an ingestible composition.

In certain aspects, the disclosure provides uses of the flavor-modifying compounds of the first aspect to reduce an astringent taste (such as a lingering aftertaste or a licorice aftertaste of a high-intensity sweetener) of an ingestible composition. In certain related aspects, the disclosure provides methods of reducing an astringent taste of an ingestible composition, the method comprising introducing a flavor-modifying compound of the first aspect to an ingestible composition.

In certain aspects, the disclosure provides uses of the flavor-modifying compounds of the first aspect to enhance mouthfeel of an ingestible composition. In certain related aspects, the disclosure provides methods of enhancing a mouthfeel of an ingestible composition, the method comprising introducing a flavor-modifying compound of the first aspect to an ingestible composition.

The preceding uses and methods involve ingestible compositions. In addition to the features of those ingestible compositions set forth above, the ingestible compositions can incorporate any features or combinations of features set forth below.

### Ingestible Compositions

In certain aspects, the disclosure provides ingestible compositions comprising flavor-modifying compounds according to the embodiments set forth above. When introduced to or used in the ingestible composition, the flavor-modifying compounds are used or introduced to in the ingestible composition at a concentration ranging from 0.01 ppm to 1000 ppm, or from 0.01 ppm to 900 ppm, or from 0.01 ppm to 800 ppm, or from 0.01 ppm to 700 ppm, or from 0.01 ppm to 600 ppm, or from 0.1 ppm to 500 ppm, or from 0.1 ppm to 400 ppm, or from 0.1 ppm to 300 ppm, or from 0.1 ppm to 200 ppm, or from 1 ppm to 100 ppm, or from 1 ppm to 80 ppm, or from 1 ppm to 60 ppm, or from 1 ppm to 50 ppm, or from 1 ppm to 40 ppm.

In some embodiments, the ingestible composition comprises one or more bitter tastants. In some embodiments, the bitter tastant is a high-intensity sweetener, such as acesulfame potassium, aspartame, neotame, cyclamate, saccharin, sucralose, steviol glycodises (such as rebaudioside A, rebaudioside B, rebaudioside M, rebaudioside D, or rebaudioside E), and mogrosides (such as mogroside III, mogroside IV, mogroside V, siamenoside I, isomogroside V, mogroside IVE, isomogroside IV, mogroside III_{E}, 11-oxomogroside V, or the 1,6-α isomer of siamenoside I). Thus, flavor-modifying compounds may be suitable used in reduced-sugar or zero-sugar products to reduce the bitterness imparted by the low-calorie or zero-calorie sweeteners.

In some embodiments, the bitter tastant is a potassium salt, such as potassium chloride, which is often used as a partial or complete replacement of sodium chloride in certain low-sodium or zero-sodium foods. Thus, flavor-modifying compounds may be suitable used in such products to reduce the bitterness imparted by potassium salts.

In some embodiments, the bitter tastant a non-animal protein, such as a plant protein, an algal protein, or a mycoprotein. In some embodiments, the ingestible composition comprises a plant protein. Non-limiting examples of plant proteins include pea protein, soy protein, almond protein, cashew protein, canola (rapeseed) protein, chickpea protein, fava protein, sunflower protein, wheat protein, oat protein, barley protein, and potato protein. Such non-animal proteins are often used as a partial or full replacement of animal proteins in dairy analogues and meat analogues. Thus, flavor-modifying compounds may be suitable used in such products to reduce the bitterness imparted by the non-animal proteins. By blocking the bitterness of such proteins, the flavor-modifying compounds can reduce the perceived cereal notes and green notes experienced by consumers.

In some embodiments, the bitter tastant is caffeine, quinine, green tea, catechins, polyphenols (such as a polyphenol antioxidants), tannins, green robusta coffee extract, green coffee extract, menthol, and the like. Such compounds commonly occur in various natural foods products, such as tea and coffee, and in packaged food products, such as instant tea, instant coffee, packaged beverages, and the like. When one or more such bitter tastants are present, the flavor-modifying compounds are suitably used to block the bitterness of such compounds and improve the perceived taste of the product to consumers.

In some embodiments, the bitter tastant is a pharmaceutical compound. Non-limiting examples of pharmaceutical compounds having a bitter taste include atropine, brinzolamide, chloramphenicol, chloroquine, clindamycin, dexamethasone, digoxin, diltiazem, diphenhydramine, docusate, dorzolamide, doxepin, doxylamine, enalapril, erythromycin, esomeprazole, famotidine, gabapentin, ginkgolide A, guaifenesin, L-histidine, lomefloxacin, methylprednisolone, ofloxacin, oleuropein, oxyphenonium, pirenzepine, prednisone, ranitidine, trapidil, trimethoprim, and cetirizine. When one or more such pharmaceutical compounds are used in an oral pharmaceutical formulation, the flavor-modifying compounds are suitably used to block the bitterness of such compounds and improve the perceived taste of the pharmaceutical product to consumers.

In some embodiments, the bitter tastant is an oral care ingredient. Many oral care ingredients impart a bitter off taste, which must be masked or blocked to improve consumer acceptance of the product. Non-limiting examples of such oral care ingredients include menthol, menthol analogues, mint extracts, sodium bicarbonate, alkali metal salts of peroxymonosulfate (potassium peroxymonosulfate), cetylpyridinium chloride, lauramidopropyl betaine, cocamidopropyl betaine, arginine, hydrogen peroxide, chlorhexidine gluconate, potassium nitrate, pentasodium triphosphate, tetrasodium pyrophosphate, stannous fluoride, thymol, methyl salicylate, eucalyptol, thymol, cubebol, and any combination thereof. When one or more such oral care compounds are used in oral care products, the flavor-modifying compounds are suitably used to block the bitterness of such compounds and improve the perceived taste of the oral care product to consumers.

In some embodiments, the bitter tastant is a bitter agent found in citrus, such as limonin, nomelin, or naringin. Many citrus-containing preparatuions impart a bitter off taste, which must be masked or blocked to improve consumer acceptance of the product. This bitter off-taste can, in some cases, be attributed to the citrus greening disease, which cases citrus fruits to turn green before fully ripening. When one or more such citrus bitter agents are present in a product, the flavor-modifying compounds are suitably used to block the bitterness of such compounds and improve the perceived taste of the citrus product to consumers.

In some embodiments, the ingestible compositions comprise one or more flavanones or dihydrochalcones. In certain embodiments, such flavanones work synergistically with the flavor-modifying compounds disclosed herein to reduce bitterness, enhance the perception of sweetness, or reduce sourness. In some embodiments, the flavanone is eriodictyol, hesperetin, hesperidin, homoeriodictyol, naringenin, hesperitin dihydrochalcone, phloretin, or any combination thereof. In some further embodiments, the flavanone is eriodictyol. In some other embodiments, the flavanone is homoeriodictyol. Such flavanones can be present in the ingestible composition at any suitable concentration, such as at a concentration ranging from 0.01 ppm to 1000 ppm, or from 0.01 ppm to 900 ppm, or from 0.01 ppm to 800 ppm, or from 0.01 ppm to 700 ppm, or from 0.01 ppm to 600 ppm, or from 0.1 ppm to 500 ppm, or from 0.1 ppm to 400 ppm, or from 0.1 ppm to 300 ppm, or from 0.1 ppm to 200 ppm, or from 1 ppm to 100 ppm, or from 1 ppm to 80 ppm, or from 1 ppm to 60 ppm, or from 1 ppm to 50 ppm, or from 1 ppm to 40 ppm.

In some embodiments, the ingestible composition includes a sweetener or a combination of sweeteners. In some embodiments, the sweetener is a common saccharide sweeteners, such as sucrose, fructose, glucose, and sweetener compositions comprising natural sugars, such as corn syrup (including high fructose corn syrup) or other syrups or sweetener concentrates derived from natural fruit and vegetable sources. In some embodiments, the sweetener is sucrose, fructose, or a combination thereof. In some embodiments, the sweetener is sucrose. In some other embodiments, the sweetener is selected from rare natural sugars including D-allose, D-psicose, L-ribose, D-tagatose, L-glucose, L-fucose, L-arbinose, D-turanose, and D-leucrose. In some embodiments, the sweetener is selected from semi-synthetic “sugar alcohol” sweeteners such as erythritol, isomalt, lactitol, mannitol, sorbitol, xylitol, maltodextrin, and the like. In some embodiments, the sweetener is selected from artificial sweeteners such as aspartame, saccharin, acesulfame-K, cyclamate, sucralose, and alitame. In some embodiments, the sweetener is selected from the group consisting of cyclamic acid, mogroside, tagatose, maltose, galactose, mannose, sucrose, fructose, lactose, allulose, neotame and other aspartame derivatives, glucose, D-tryptophan, glycine, maltitol, lactitol, isomalt, hydrogenated glucose syrup (HGS), hydrogenated starch hydrolyzate (HSH), stevioside, rebaudioside A, other sweet Stevia-based glycosides, chemically modified steviol glycosides (such as glucosylated steviol glycosides), mogrosides, chemically modified mogrosides (such as glucosylated mogrosides), carrelame and other guanidine-based sweeteners. In some embodiments, the sweetener is a combination of two or more of the sweeteners set forth in this paragraph. In some embodiments, the sweetener may combinations of two, three, four or five sweeteners as disclosed herein. In some embodiments, the sweetener may be a sugar. In some embodiments, the sweetener may be a combination of one or more sugars and other natural and artificial sweeteners. In some embodiments, the sweetener is a sugar. In some embodiments, the sugar is cane sugar. In some embodiments, the sugar is beet sugar. In some embodiments, the sugar may be sucrose, fructose, glucose or combinations thereof. In some embodiments, the sugar may be sucrose. In some embodiments, the sugar may be a combination of fructose and glucose.

The sweetener can also include, for example, sweetener compositions comprising one or more natural or synthetic carbohydrate, such as corn syrup, high fructose corn syrup, high maltose corn syrup, glucose syrup, sucralose syrup, hydrogenated glucose syrup (HGS), hydrogenated starch hydrolyzate (HSH), or other syrups or sweetener concentrates derived from natural fruit and vegetable sources, or semi-synthetic “sugar alcohol” sweeteners such as polyols. Non-limiting examples of polyols in some embodiments include erythritol, maltitol, mannitol, sorbitol, lactitol, xylitol, isomalt, propylene glycol, glycerol (glycerin), threitol, galactitol, palatinose, reduced isomalto-oligosaccharides, reduced xylo-oligosaccharides, reduced gentio-oligosaccharides, reduced maltose syrup, reduced glucose syrup, isomaltulose, maltodextrin, and the like, and sugar alcohols or any other carbohydrates or combinations thereof capable of being reduced which do not adversely affect taste.

The sweetener may be a natural or synthetic sweetener that includes, but is not limited to, agave inulin, agave nectar, agave syrup, amazake, brazzein, brown rice syrup, coconut crystals, coconut sugars, coconut syrup, date sugar, fructans (also referred to as inulin fiber, fructo-oligosaccharides, or oligo-fructose), green stevia powder, Stevia rebaudiana, rebaudioside A, rebaudioside B, rebaudioside C, rebaudioside D, rebaudioside E, rebaudioside F, rebaudioside I, rebaudioside H, rebaudioside L, rebaudioside K, rebaudioside J, rebaudioside N, rebaudioside O, rebaudioside M and other sweet Stevia-based glycosides, stevioside, stevioside extracts, honey, Jerusalem artichoke syrup, licorice root, luo han guo (fruit, powder, or extracts), lucuma (fruit, powder, or extracts), maple sap (including, for example, sap extracted from Acer saccharum, Acer nigrum, Acer rubrum, Acer saccharinum, Acer platanoides, Acer negundo, Acer macrophyllum, Acer grandidentatum, Acer glabrum, Acer mono), maple syrup, maple sugar, walnut sap (including, for example, sap extracted from Juglans cinerea, Juglans nigra, Juglans ailatifolia, Juglans regia), birch sap (including, for example, sap extracted from Betula papyrifera, Betula alleghaniensis, Betula lenta, Betula nigra, Betula populifolia, Betula pendula), sycamore sap (such as, for example, sap extracted from Platanus occidentalis), ironwood sap (such as, for example, sap extracted from Ostrya virginiana), mascobado, molasses (such as, for example, blackstrap molasses), molasses sugar, monatin, monellin, cane sugar (also referred to as natural sugar, unrefined cane sugar, or sucrose), palm sugar, panocha, piloncillo, rapadura, raw sugar, rice syrup, sorghum, sorghum syrup, cassava syrup (also referred to as tapioca syrup), thaumatin, yacon root, malt syrup, barley malt syrup, barley malt powder, beet sugar, cane sugar, crystalline juice crystals, caramel, carbitol, carob syrup, castor sugar, hydrogenated starch hydrolates, hydrolyzed can juice, hydrolyzed starch, invert sugar, anethole, arabinogalactan, arrope, syrup, P-4000, acesulfame potassium (also referred to as acesulfame K or ace-K), alitame (also referred to as aclame), advantame, aspartame, baiyunoside, neotame, benzamide derivatives, bernadame, canderel, carrelame and other guanidine-based sweeteners, vegetable fiber, corn sugar, coupling sugars, curculin, cyclamates, cyclocarioside I, demerara, dextran, dextrin, diastatic malt, dulcin, sucrol, valzin, dulcoside A, dulcoside B, emulin, enoxolone, maltodextrin, saccharin, estragole, ethyl maltol, glucin, gluconic acid, glucono-lactone, glucosamine, glucoronic acid, glycerol, glycine, glycyphillin, glycyrrhizin, glycyrrhetic acid monoglucuronide, golden sugar, yellow sugar, golden syrup, granulated sugar, gynostemma, hernandulcin, isomerized liquid sugars, jallab, chicory root dietary fiber, kynurenine derivatives (including N′-formyl-kynurenine, N′-acetyl-kynurenine, 6-chloro-kynurenine), galactitol, litesse, ligicane, lycasin, lugduname, guanidine, falernum, mabinlin I, mabinlin II, maltol, maltisorb, maltodextrin, maltotriol, mannosamine, miraculin, mizuame, mogrosides (including, for example, mogroside IV, mogroside V, and neomogroside), mukurozioside, nano sugar, naringin dihydrochalcone, neohesperidine dihydrochalcone, nib sugar, nigero-oligosaccharide, norbu, orgeat syrup, osladin, pekmez, pentadin, periandrin I, perillaldehyde, perillartine, petphyllum, phenylalanine, phlomisoside I, phlorodizin, phyllodulcin, polyglycitol syrups, polypodoside A, pterocaryoside A, pterocaryoside B, rebiana, refiners syrup, rub syrup, rubusoside, selligueain A, shugr, siamenoside I, siraitia grosvenorii, soybean oligosaccharide, Splenda, SRI oxime V, steviol glycoside, steviolbioside, stevioside, strogins 1, 2, and 4, sucronic acid, sucrononate, sugar, suosan, phloridzin, superaspartame, tetrasaccharide, threitol, treacle, trilobtain, tryptophan and derivatives (6-trifluoromethyl-tryptophan, 6-chloro-D-tryptophan), vanilla sugar, volemitol, birch syrup, aspartame-acesulfame, assugrin, and combinations or blends of any two or more thereof.

Additional sweeteners also include combinations of any two or more of any of the aforementioned sweeteners. In some embodiments, the sweetener may comprise combinations of two, three, four or five sweeteners as disclosed herein. In some embodiments, the sweetener may be a sugar. In some embodiments, the sweetener may be a combination of one or more sugars and other natural and artificial sweeteners. In some embodiments, the sweetener is a caloric sweetener, such as sucrose, fructose, xylitol, erythritol, or combinations thereof. In some embodiments, the ingestible compositions are free (or, in some embodiments) substantially free of stevia-derived sweeteners, such as steviol glycosides, glucosylated steviol glycosides, or rebaudiosides.

In some embodiments, the sweetener is sucrose. In some other embodiments, the sweetener is a steviol glycoside.

In some embodiments, the ingestible composition comprises a sour tastant, such as an organic acid. Non-limiting examples of such organic acids include acetic acid, malonic acid, citric acid, lactic acid, and the like.

The ingestible compositions can, in certain embodiments, comprise any additional ingredients or combination of ingredients as are commonly used in food and beverage products, including, but not limited to:
acids, including, for example citric acid, phosphoric acid, ascorbic acid, sodium acid sulfate, lactic acid, or tartaric acid;
bitter ingredients, including, for example caffeine, quinine, green tea, catechins, polyphenols, green robusta coffee extract, green coffee extract, potassium chloride, menthol, or proteins (such as proteins and protein isolates derived from plants, algae, or fungi); coloring agents, including, for example caramel color, Red #40, Yellow #5, Yellow #6, Blue #1, Red #3, purple carrot, black carrot juice, purple sweet potato, vegetable juice, fruit juice, beta carotene, turmeric curcumin, or titanium dioxide;
preservatives, including, for example sodium benzoate, potassium benzoate, potassium sorbate, sodium metabisulfate, sorbic acid, or benzoic acid;
antioxidants including, for example ascorbic acid, calcium disodium EDTA, alpha tocopherols, mixed tocopherols, rosemary extract, grape seed extract, resveratrol, or sodium hexametaphosphate;
vitamins or functional ingredients including, for example resveratrol, Co-Q10, omega 3 fatty acids, theanine, choline chloride (citocoline), fibersol, inulin (chicory root), taurine, panax ginseng extract, guanana extract, ginger extract, L-phenylalanine, L-carnitine, L-tartrate, D-glucoronolactone, inositol, bioflavonoids, Echinacea, ginko biloba, yerba mate, flax seed oil, garcinia cambogia rind extract, white tea extract, ribose, milk thistle extract, grape seed extract, pyrodixine HCl (vitamin B6), cyanoobalamin (vitamin B12), niacinamide (vitamin B3), biotin, calcium lactate, calcium pantothenate (pantothenic acid), calcium phosphate, calcium carbonate, chromium chloride, chromium polynicotinate, cupric sulfate, folic acid, ferric pyrophosphate, iron, magnesium lactate, magnesium carbonate, magnesium sulfate, monopotassium phosphate, monosodium phosphate, phosphorus, potassium iodide, potassium phosphate, riboflavin, sodium sulfate, sodium gluconate, sodium polyphosphate, sodium bicarbonate, thiamine mononitrate, vitamin D3, vitamin A palmitate, zinc gluconate, zinc lactate, or zinc sulfate;
clouding agents, including, for example ester gun, brominated vegetable oil (BVO), or sucrose acetate isobutyrate (SAIB);
buffers, including, for example sodium citrate, potassium citrate, or salt;
flavors, including, for example propylene glycol, ethyl alcohol, glycerine, gum Arabic (gum acacia), maltodextrin, modified corn starch, dextrose, natural flavor, natural flavor with other natural flavors (natural flavor WONF), natural and artificial flavors, artificial flavor, silicon dioxide, magnesium carbonate, or tricalcium phosphate; or starches and stabilizers, including, for example pectin, xanthan gum, carboxylmethylcellulose (CMC), polysorbate 60, polysorbate 80, medium chain triglycerides, cellulose gel, cellulose gum, sodium caseinate, modified food starch, gum Arabic (gum acacia), inulin, or carrageenan.

The ingestible compositions can have any suitable pH. In some embodiments, the flavor-modifying compounds enhance the sweetness of a sweetener under a broad range of pH, e.g., from lower pH to neutral pH. The lower and neutral pH includes, but is not limited to, a pH from 1.5 to 9.0, or from 2.5 to 8.5; from 3.0 to 8.0; from 3.5 to 7.5; and from 4.0 to 7. In certain embodiments, compounds as disclosed and described herein, individually or in combination, can enhance the perceived sweetness of a fixed concentration of a sweetener in taste tests at a compound concentration of 50 μM, 40 μM, 30 μM, 20 μM, or 10 μM at both low to neutral pH value. In certain embodiments, the enhancement factor of the compounds as disclosed and described herein, individually or in combination, at the lower pH is substantially similar to the enhancement factor of the compounds at neutral pH. Such consistent sweet enhancing property under a broad range of pH allow a broad use in a wide variety of foods and beverages of the compounds as disclosed and described herein, individually or in combination.

In some embodiments, the ingestible composition comprises a flavoring. Any suitable flavoring can be used. In some embodiments, the flavoring comprises synthetic flavor oils and flavoring aromatics or oils, oleoresins and extracts derived from plants, leaves, flowers, fruits, and so forth, or combinations thereof. Non-limiting examples of flavor oils include spearmint oil, cinnamon oil, oil of wintergreen (methyl salicylate), peppermint oil, Japanese mint oil, clove oil, bay oil, anise oil, eucalyptus oil, thyme oil, cedar leaf oil, oil of nutmeg, allspice, oil of sage, mace, oil of bitter almonds, and cassia oil. Non-limiting examples of other flavors include natural and synthetic fruit flavors such as vanilla, and citrus oils including lemon, orange, lime, grapefruit, yazu, sudachi, and fruit essences including apple, pear, peach, grape, blueberry, strawberry, raspberry, cherry, plum, pineapple, watermelon, apricot, banana, melon, apricot, ume, cherry, raspberry, blackberry, tropical fruit, mango, mangosteen, pomegranate, papaya and so forth. Other potential flavors include a milk flavor, a butter flavor, a cheese flavor, a cream flavor, and a yogurt flavor; a vanilla flavor; tea or coffee flavors, such as a green tea flavor, a oolong tea flavor, a tea flavor, a cocoa flavor, a chocolate flavor, and a coffee flavor; mint flavors, such as a peppermint flavor, a spearmint flavor, and a Japanese mint flavor; spicy flavors, such as an asafetida flavor, an ajowan flavor, an anise flavor, an angelica flavor, a fennel flavor, an allspice flavor, a cinnamon flavor, a chamomile flavor, a mustard flavor, a cardamom flavor, a caraway flavor, a cumin flavor, a clove flavor, a pepper flavor, a coriander flavor, a sassafras flavor, a savory flavor, a Zanthoxyli Fructus flavor, a perilla flavor, a juniper berry flavor, a ginger flavor, a star anise flavor, a horseradish flavor, a thyme flavor, a tarragon flavor, a dill flavor, a capsicum flavor, a nutmeg flavor, a basil flavor, a marjoram flavor, a rosemary flavor, a bayleaf flavor, and a wasabi (Japanese horseradish) flavor; alcoholic flavors, such as a wine flavor, a whisky flavor, a brandy flavor, a rum flavor, a gin flavor, and a liqueur flavor; floral flavors; and vegetable flavors, such as an onion flavor, a garlic flavor, a cabbage flavor, a carrot flavor, a celery flavor, mushroom flavor, and a tomato flavor. These flavoring agents may be used in liquid or solid form and may be used individually or in admixture. In the context of dairy or dairy analog products, the most commonly used flavor agents are agents that impart flavors such as vanilla, French vanilla, chocolate, banana, lemon, hazelnut, coconut, almond, strawberry, mocha, coffee, tea, chai, cinnamon, caramel, cream, brown sugar, toffee, pecan, butter pecan, toffee, Irish crème, white chocolate, raspberry, pumpkin pie spice, peppermint, or any combination thereof.

In some embodiments, the ingestible composition comprises vanillin or a vanillin analogue, which impart a vanilla flavor to the flavoring. In some further embodiments, the ingestible composition comprises one or more lactones, which impart a creamy flavor to the composition.

In some embodiments, the ingestible composition comprises a yeast extract, such as a yeast lysate. Such extracts can be obtained from any suitable yeast strain, where such extracts are suitable for human consumption. Non-limiting examples of such yeasts include: yeasts of the genus Saccharomyces, such as Saccharomyces cerevisiae or Saccharomyces pastorianus; yeasts of the genus Candida, such as Candida utilis; yeasts of the genus Kluyveromyces, such as Kluyveromyces lactis or Kluyveromyces marxianus; yeasts of the genus Pichia such as Pichia pastoris; yeasts of the genus Debaryomyces such as Debaryomyces hansenii; and yeasts of the genus Zygosaccharomyces such as Zygosaccharomyces mellis. In some embodiments, the yeast is a yeast collected after brewing beer, sake, or the like. In some embodiments, the yeast is a yeast subjected to drying treatment (dried yeast) after collection.

Such extracts can be produced by any suitable means. In general, yeast extracts or lysates are made by extracting the contents of the yeast cells from the cell wall material. In many instances, the digestive enzymes in the cells (or additional enzymes added to the composition) break down the proteins and polynucleotides in the yeast to amino acids, oligopeptides (for example, from 2 to 10 peptides), nucleotides, oligonucleotides (from 2 to nucleotides), and mixtures thereof. A yeast lysate can be prepared by lysing a yeast. For example, in some embodiments, the yeast after culture is crushed or lysed by an enzymatic decomposition method, a self-digestion method, an alkaline extraction method, a hot water extraction method, an acid decomposition method, an ultrasonic crushing method, crushing with a homogenizer, a freezing-thawing method, or the like (two or more thereof may be used in combination), whereby a yeast lysate is obtained. Yeast may be cultured by a conventional method. In some embodiments, the yeast after culture is heat-treated and then treated with a lytic enzyme to obtain an enzyme lysate. The conditions for the heat treatment are, for example, 80° C. to 90° C. for 5 minutes to 30 minutes. As the lytic enzyme used for the enzymatic decomposition method, various enzymes can be used as long as they can lyse the cell wall of yeast. The reaction conditions may be set so as to be optimum or suitable for the lytic enzyme(s) to be used, and specific examples thereof can include a temperature of 50° C. to 60° C., and a pH of 7.0 to 8.0. The reaction time is also not particularly limited, and can be, for example, 3 hours to 5 hours.

Compositions comprising yeast lysate can be obtained from a variety of commercial sources. For example, in some embodiments, the yeast lysate is provides by the flavoring additive sold under the name MODUMAX (DSM Food Specialties BV, Delft, Netherlands).

In some embodiments, the ingestible composition comprises a sweetness enhancer. Any suitable sweetness enhancer can be used in the ingestible compositions disclosed herein, including synthetic sweetness enhancers, natural sweetness enhancers, or any combinations thereof.

Examples of suitable synthetic sweetness enhancers include, but are not limited to, N-(1-((4-amino-2,2-dioxo-1H-benzo[c][1,2,6]thiadiazin-5-yl)oxy)-2-methylpropan-2-yl)isonicotinamide, or any of its comestbly acceptable salts, 3-hydroxybenzoic acid, or any compounds set forth in U.S. Pat. Nos. 8,541,421; 8,815,956; 9,834,544; 8,592,592; 8,877,922; 9,000,054; and 9,000,051, as well as U.S. Patent Application Publication No. 2017/0119032.

Suitable examples of natural sweetness enhancers include, but are not limited to, hesperetin dihydrochalcone, hesperetin dihydrochalcone-4′-O′glucoside, neohesperetin dihydrochalcone, brazzein, hesperidin, phyllodulcin, naringenin, naringin, phloretin, glucosylated steviol glycosides, (2R,3R)-3-acetoxy-5,7,4′-trihydroxyflavanone, (2R,3R)-3-acetoxy-5,7,3′-trihydroxy-4′-methoxyflavanone, rubusosides, thaumatin, monellin, miraculin, glycyrrhizin and comestible acceptable salts thereof (such as the mono-ammonium salt), naringin dihydrochalcone, myricetin, nobiletin, polymethoxyflavones, mixed methoxy- and hydroxyflavones, quercetin, certain amino acids, and the like. As used herein, the term “glucosylated steviol glycoside” refers to the product of enzymatically glucosylating natural steviol glycoside compounds. The glucosylation generally occurs through a glycosidic bond, such as an α-1,2 bond, an α-1,4 bond, an α-1.6 bond, a β-1,2 bond, a β-1,4 bond, a β-1,6 bond, and so forth.

In some embodiments of any of the preceding embodiments, the ingestible composition comprises 3-((4-amino-2,2-dioxo-1H-benzo[c][1,2,6]thiadiazin-5-yl)oxy)-2,2-dimethyl-N-propyl-propanamide, N-(1-((4-amino-2,2-dioxo-1H-benzo[c][1,2,6]-thiadiazin-5-yl)oxy)-2-methyl-propan-2-yl)isonicotinamide, or a comestibly acceptable salt thereof. In some embodiments, the ingestible composition comprises N-(1-((4-amino-2,2-dioxo-1H-benzo[c][1,2,6]thiadiazin-5-yl)oxy)-2-methyl-propan-2-yl) isonicotinamide, or a comestbly acceptable salt thereof. In some embodiments, the ingestible composition comprises N-(1-((4-amino-2,2-dioxo-1H-benzo[c][1,2,6]thiadiazin-5-yl)oxy)-2-methyl-propan-2-yl)isonicotinamide.

In some embodiments, the ingestible composition comprises one or more umami enhancing compounds. Such umami enhancing compounds include, but are not limited to, naturally derived compounds, or synthetic compounds, such as any compounds set forth in U.S. Pat. Nos. 8,735,081; 8,124,121; and 8,968,708. In some embodiments, the umami-enhancing compound is (2R,4R)-1,2,4-trihydroxy-heptadec-16-ene, (2R,4R)-1,2,4-trihydroxyheptadec-16-yne, or a mixture thereof. In some embodiments, the umami-enhancing compound is (3R,5S)-1-(4-hydroxy-3-methoxyphenyl) decane-3,5-diol diacetate. In some embodiments, the umami-enhancing compound is N-(heptan-4-yl)benzo-[d][1,3]dioxole-5-carboxamide.

In some further embodiments, the ingestible composition comprises one or more cooling enhancing compounds. Such cooling enhancing compounds include, but are not limited to, naturally derived compounds, such as menthol or analogs thereof, or synthetic compounds, such as any compounds set forth in U.S. Pat. Nos. 9,394,287 and 10,421,727. Non-limiting examples include N-ethyl-N-(thiophen-2-ylmethyl)-2-(p-tolyloxy)acetamide, N-(1H-pyrazol-3-yl)-N-(thiophen-2-ylmethyl)-2-(p-tolyloxy)acetamide, 2-(4-fluorophenoxy)-N-(1H-pyrazol-3-yl)-N-(thiophen-2-ylmethyl) acetamide, 2-(2-hydroxy-4-methylphenoxy)-N-(1H-pyrazol-3-yl)-N-(thiophen-2-ylmethyl)-acetamide, 2-((2,3-dihydro-1H-inden-5-yl)oxy)-N-(1H-pyrazol-3-yl)-N-(thiophen-2-ylmethyl)-acetamide, 2-((2,3-dihydro-1H-inden-5-yl)oxy)-N-(1H-pyrazol-3-yl)-N-(thiazol-5-ylmethyl)-acetamide, 2-((5-methoxy benzofuran-2-yl)oxy)-N-(1H-pyrazol-3-yl)-N-(thiophen-2-ylmethyl)-acetamide, (E/Z)-2-methyl-2-butenal, (E/Z)-2-isopropyl-5-methyl-2-hexenal, phloretin, naringenin, and any combinations thereof.

In some further embodiments, the ingestible composition comprises one or more bitterness blocking or bitter masking compounds. Such bitterness blocking compounds or bitter masking include, but are not limited to, naturally derived compounds or synthetic compounds, such as any compounds set forth in U.S. Pat. Nos. 8,076,491; 8,445,692; and 9,247,759. Non-limiting examples include 3-(1-((3,5-dimethylisoxazol-4-yl)-methyl)-1H-pyrazol-4-yl)-1-(3-hydroxybenzyl)-imidazolidine-2,4-dione, 4-(2,2,3-trimethyl-cyclopentyl)butanoic acid, 3-hydroxydihydrocostunolide, 3β-hydroxypelenolide, probenecid, sakuranetin, 6-methoxysakuranetin, jaceosidin, 4′-fluoro-6-methoxyflavonone, 6,3′-dimethoxyflavonone, 6-methoxyflavonone, γ-aminobutyric acid, Nα,Nα-bis(carbomethyl)-L-lysine, (+/−) abscisic acid, sodium gluconate, monosodium glutamate, sodium acetate, homoeriodictyol, sterubin, eriodictyol, 2,4, dihydrobenzoic acid, neodiosmin, 1-carboxymethyl-5-hydroxy-2-hydroxymethylpyridinium, flavan-3-spiro-C-glycosides, poly-γ-glutamic acid, α,α-trehalose, taurine, (2)-gingerdione, 2,4,-dihydroxybenzoic acid, L-theanine, enterodiol, lariciresinol, enterolactone, matairesinol, and any combinations thereof.

In some further embodiments, the ingestible composition comprises one or more sour taste modulating compounds.

In some further embodiments, the ingestible composition comprises one or more mouthfeel modifying compounds. Such mouthfeel modifying compounds include, but are not limited to, tannins, cellulosic materials, bamboo powder, and the like.

In some further embodiments, the ingestible composition comprises one or more flavor masking compounds. Such flavor masking compounds include, but are not limited to, cellulosic materials, materials extracted from fungus, materials extracted from plants, citric acid, carbonic acid (or carbonates), and the like.

### Flavored Products

In certain aspects, the disclosure provides flavored products comprising any ingestible compositions of the preceding aspects. In some embodiments, the flavored products are beverage products, such as soda, flavored water, tea, and the like. In some other embodiments, the flavored products are food products, such as yogurt. In some embodiments, the flavored products are oral care products, such as toothpaste, mouthwash, dentrifrices, whitening agents and the like.

In embodiments where the flavored product is a beverage, the beverage may be selected from the group consisting of enhanced sparkling beverages, colas, lemon-lime flavored sparkling beverages, orange flavored sparkling beverages, grape flavored sparkling beverages, strawberry flavored sparkling beverages, pineapple flavored sparkling beverages, ginger-ales, root beers, fruit juices, fruit-flavored juices, juice drinks, nectars, vegetable juices, vegetable-flavored juices, sports drinks, energy drinks, enhanced water drinks, enhanced water with vitamins, near water drinks, coconut waters, tea type drinks, coffees, cocoa drinks, beverages containing milk components, beverages containing cereal extracts and smoothies. In some embodiments, the beverage may be a soft drink.

In certain embodiments of any aspects and embodiments set forth herein that refer to an flavored product, the flavored product is a non-naturally-occurring product, such as a packaged food or beverage product.

Further non-limiting examples of food and beverage products or formulations include sweet coatings, frostings, or glazes for such products or any entity included in the Soup category, the Dried Processed Food category, the Beverage category, the Ready Meal category, the Canned or Preserved Food category, the Frozen Processed Food category, the Chilled Processed Food category, the Snack Food category, the Baked Goods category, the Confectionery category, the Dairy Product category, the Ice Cream category, the Meal Replacement category, the Pasta and Noodle category, and the Sauces, Dressings, Condiments category, the Baby Food category, and/or the Spreads category.

In general, the Soup category refers to canned/preserved, dehydrated, instant, chilled, UHT and frozen soup. For the purpose of this definition soup(s) means a food prepared from meat, poultry, fish, vegetables, grains, fruit and other ingredients, cooked in a liquid which may include visible pieces of some or all of these ingredients. It may be clear (as a broth) or thick (as a chowder), smooth, pureed or chunky, ready-to-serve, semi-condensed or condensed and may be served hot or cold, as a first course or as the main course of a meal or as a between meal snack (sipped like a beverage). Soup may be used as an ingredient for preparing other meal components and may range from broths (consommé) to sauces (cream or cheese-based soups).

The Dehydrated and Culinary Food Category usually means: (i) Cooking aid products such as: powders, granules, pastes, concentrated liquid products, including concentrated bouillon, bouillon and bouillon like products in pressed cubes, tablets or powder or granulated form, which are sold separately as a finished product or as an ingredient within a product, sauces and recipe mixes (regardless of technology); (ii) Meal solutions products such as: dehydrated and freeze dried soups, including dehydrated soup mixes, dehydrated instant soups, dehydrated ready-to-cook soups, dehydrated or ambient preparations of ready-made dishes, meals and single serve entrees including pasta, potato and rice dishes; and (iii) Meal embellishment products such as: condiments, marinades, salad dressings, salad toppings, dips, breading, batter mixes, shelf stable spreads, barbecue sauces, liquid recipe mixes, concentrates, sauces or sauce mixes, including recipe mixes for salad, sold as a finished product or as an ingredient within a product, whether dehydrated, liquid or frozen.

The Beverage category usually means beverages, beverage mixes and concentrates, including but not limited to, carbonated and non-carbonated beverages, alcoholic and non-alcoholic beverages, ready to drink beverages, liquid concentrate formulations for preparing beverages such as sodas, and dry powdered beverage precursor mixes. The Beverage category also includes the alcoholic drinks, the soft drinks, sports drinks, isotonic beverages, and hot drinks. The alcoholic drinks include, but are not limited to beer, cider/perry, FABs, wine, and spirits. The soft drinks include, but are not limited to carbonates, such as colas and non-cola carbonates; fruit juice, such as juice, nectars, juice drinks and fruit flavored drinks; bottled water, which includes sparkling water, spring water and purified/table water; functional drinks, which can be carbonated or still and include sport, energy or elixir drinks; concentrates, such as liquid and powder concentrates in ready to drink measure. The drinks, either hot or cold, include, but are not limited to coffee or ice coffee, such as fresh, instant, and combined coffee; tea or ice tea, such as black, green, white, oolong, and flavored tea; and other drinks including flavor-, malt- or plant-based powders, granules, blocks or tablets mixed with milk or water.

The Snack Food category generally refers to any food that can be a light informal meal including, but not limited to Sweet and savory snacks and snack bars. Examples of snack food include, but are not limited to fruit snacks, chips/crisps, extruded snacks, tortilla/corn chips, popcorn, pretzels, nuts and other sweet and savory snacks. Examples of snack bars include, but are not limited to granola/muesli bars, breakfast bars, energy bars, fruit bars and other snack bars.

The Baked Goods category generally refers to any edible product the process of preparing which involves exposure to heat or excessive sunlight. Examples of baked goods include, but are not limited to bread, buns, cookies, muffins, cereal, toaster pastries, pastries, waffles, tortillas, biscuits, pies, bagels, tarts, quiches, cake, any baked foods, and any combination thereof.

The Ice Cream category generally refers to frozen dessert containing cream and sugar and flavoring. Examples of ice cream include, but are not limited to: impulse ice cream; take-home ice cream; frozen yoghurt and artisanal ice cream; soy, oat, bean (e.g., red bean and mung bean), and rice-based ice creams.

The Confectionery category generally refers to edible product that is sweet to the taste. Examples of confectionery include, but are not limited to candies, gelatins, chocolate confectionery, sugar confectionery, gum, and the likes and any combination products.

The Meal Replacement category generally refers to any food intended to replace the normal meals, particularly for people having health or fitness concerns. Examples of meal replacement include, but are not limited to slimming products and convalescence products.

The Ready Meal category generally refers to any food that can be served as meal without extensive preparation or processing. The ready meal includes products that have had recipe “skills” added to them by the manufacturer, resulting in a high degree of readiness, completion and convenience. Examples of ready meal include, but are not limited to canned/preserved, frozen, dried, chilled ready meals; dinner mixes; frozen pizza; chilled pizza; and prepared salads.

The Pasta and Noodle category includes any pastas and/or noodles including, but not limited to canned, dried and chilled/fresh pasta; and plain, instant, chilled, frozen and snack noodles.

The Canned/Preserved Food category includes, but is not limited to canned/preserved meat and meat products, fish/seafood, vegetables, tomatoes, beans, fruit, ready meals, soup, pasta, and other canned/preserved foods.

The Frozen Processed Food category includes, but is not limited to frozen processed red meat, processed poultry, processed fish/seafood, processed vegetables, meat substitutes, processed potatoes, bakery products, desserts, ready meals, pizza, soup, noodles, and other frozen food.

The Dried Processed Food category includes, but is not limited to rice, dessert mixes, dried ready meals, dehydrated soup, instant soup, dried pasta, plain noodles, and instant noodles. The Chill Processed Food category includes, but is not limited to chilled processed meats, processed fish/seafood products, lunch kits, fresh cut fruits, ready meals, pizza, prepared salads, soup, fresh pasta and noodles.

The Sauces, Dressings and Condiments category includes, but is not limited to tomato pastes and purees, bouillon/stock cubes, herbs and spices, monosodium glutamate (MSG), table sauces, soy based sauces, pasta sauces, wet/cooking sauces, dry sauces/powder mixes, ketchup, mayonnaise, mustard, salad dressings, vinaigrettes, dips, pickled products, and other sauces, dressings and condiments

The Baby Food category includes, but is not limited to milk- or soybean-based formula; and prepared, dried and other baby food.

The Spreads category includes, but is not limited to jams and preserves, honey, chocolate spreads, nut based spreads, and yeast based spreads.

The Dairy Product category generally refers to edible product produced from mammal's milk. Examples of dairy product include, but are not limited to drinking milk products, cheese, yoghurt and sour milk drinks, and other dairy products.

Additional examples for flavored products, particularly food and beverage products or formulations, are provided as follows. Exemplary ingestible compositions include one or more confectioneries, chocolate confectionery, tablets, countlines, bagged selflines/softlines, boxed assortments, standard boxed assortments, twist wrapped miniatures, seasonal chocolate, chocolate with toys, alfajores, other chocolate confectionery, mints, standard mints, power mints, boiled sweets, pastilles, gums, jellies and chews, toffees, caramels and nougat, medicated confectionery, lollipops, liquorice, other sugar confectionery, bread, packaged/industrial bread, unpackaged/artisanal bread, pastries, cakes, packaged/industrial cakes, unpackaged/artisanal cakes, cookies, chocolate coated biscuits, sandwich biscuits, filled biscuits, savory biscuits and crackers, bread substitutes, breakfast cereals, rte cereals, family breakfast cereals, flakes, muesli, other cereals, children's breakfast cereals, hot cereals, ice cream, impulse ice cream, single portion dairy ice cream, single portion water ice cream, multi-pack dairy ice cream, multi-pack water ice cream, take-home ice cream, take-home dairy ice cream, ice cream desserts, bulk ice cream, take-home water ice cream, frozen yoghurt, artisanal ice cream, dairy products, milk, fresh/pasteurized milk, full fat fresh/pasteurized milk, semi skimmed fresh/pasteurized milk, long-life/uht milk, full fat long life/uht milk, semi skimmed long life/uht milk, fat-free long life/uht milk, goat milk, condensed/evaporated milk, plain condensed/evaporated milk, flavored, functional and other condensed milk, flavored milk drinks, dairy only flavored milk drinks, flavored milk drinks with fruit juice, soy milk, sour milk drinks, fermented dairy drinks, coffee whiteners, powder milk, flavored powder milk drinks, cream, cheese, processed cheese, spreadable processed cheese, unspreadable processed cheese, unprocessed cheese, spreadable unprocessed cheese, hard cheese, packaged hard cheese, unpackaged hard cheese, yoghurt, plain/natural yoghurt, flavored yoghurt, fruited yoghurt, probiotic yoghurt, drinking yoghurt, regular drinking yoghurt, probiotic drinking yoghurt, chilled and shelf-stable desserts, dairy-based desserts, soy-based desserts, chilled snacks, fromage frais and quark, plain fromage frais and quark, flavored fromage frais and quark, savory fromage frais and quark, sweet and savory snacks, fruit snacks, chips/crisps, extruded snacks, tortilla/corn chips, popcorn, pretzels, nuts, other sweet and savory snacks, snack bars, granola bars, breakfast bars, energy bars, fruit bars, other snack bars, meal replacement products, slimming products, convalescence drinks, ready meals, canned ready meals, frozen ready meals, dried ready meals, chilled ready meals, dinner mixes, frozen pizza, chilled pizza, soup, canned soup, dehydrated soup, instant soup, chilled soup, hot soup, frozen soup, pasta, canned pasta, dried pasta, chilled/fresh pasta, noodles, plain noodles, instant noodles, cups/bowl instant noodles, pouch instant noodles, chilled noodles, snack noodles, canned food, canned meat and meat products, canned fish/seafood, canned vegetables, canned tomatoes, canned beans, canned fruit, canned ready meals, canned soup, canned pasta, other canned foods, frozen food, frozen processed red meat, frozen processed poultry, frozen processed fish/seafood, frozen processed vegetables, frozen meat substitutes, frozen potatoes, oven baked potato chips, other oven baked potato products, non-oven frozen potatoes, frozen bakery products, frozen desserts, frozen ready meals, frozen pizza, frozen soup, frozen noodles, other frozen food, dried food, dessert mixes, dried ready meals, dehydrated soup, instant soup, dried pasta, plain noodles, instant noodles, cups/bowl instant noodles, pouch instant noodles, chilled food, chilled processed meats, chilled fish/seafood products, chilled processed fish, chilled coated fish, chilled smoked fish, chilled lunch kit, chilled ready meals, chilled pizza, chilled soup, chilled/fresh pasta, chilled noodles, oils and fats, olive oil, vegetable and seed oil, cooking fats, butter, margarine, spreadable oils and fats, functional spreadable oils and fats, sauces, dressings and condiments, tomato pastes and purees, bouillon/stock cubes, stock cubes, gravy granules, liquid stocks and fonds, herbs and spices, fermented sauces, soy based sauces, pasta sauces, wet sauces, dry sauces/powder mixes, ketchup, mayonnaise, regular mayonnaise, mustard, salad dressings, regular salad dressings, low fat salad dressings, vinaigrettes, dips, pickled products, other sauces, dressings and condiments, baby food, milk formula, standard milk formula, follow-on milk formula, toddler milk formula, hypoallergenic milk formula, prepared baby food, dried baby food, other baby food, spreads, jams and preserves, honey, chocolate spreads, nut-based spreads, and yeast-based spreads. Exemplary ingestible compositions also include confectioneries, bakery products, ice creams, dairy products, sweet and savory snacks, snack bars, meal replacement products, ready meals, soups, pastas, noodles, canned foods, frozen foods, dried foods, chilled foods, oils and fats, baby foods, or spreads or a mixture thereof. Exemplary ingestible compositions also include breakfast cereals, sweet beverages or solid or liquid concentrate compositions for preparing beverages, ideally so as to enable the reduction in concentration of previously known saccharide sweeteners, or artificial sweeteners.

Some embodiments provide a chewable composition that may or may not be intended to be swallowed. In some embodiments, the chewable composition may be gum, chewing gum, sugarized gum, sugar-free gum, functional gum, bubble gum including compounds as disclosed and described herein, individually or in combination.

Typically at least a sweet receptor modulating amount, a sweet receptor ligand modulating amount, a sweet flavor modulating amount, a sweet flavoring agent amount, a sweet flavor enhancing amount, or a therapeutically effective amount of one or more of the present compounds will be added to the ingestible composition, optionally in the presence of sweeteners so that the sweet flavor modified ingestible composition has an increased sweet taste as compared to the ingestible composition prepared without the compounds of the present invention, as judged by human beings or animals in general, or in the case of formulations testing, as judged by a majority of a panel of at least eight human taste testers, via procedures commonly known in the field.

In some embodiments, compounds as disclosed and described herein, individually or in combination, modulate the sweet taste or other taste properties of other natural or synthetic sweet tastants, and ingestible compositions made therefrom. In one embodiment, the compounds as disclosed and described herein, individually or in combination, may be used or provided in its ligand enhancing concentration(s). For example, the compounds as disclosed and described herein, individually or in combination, may be present in an amount of from 0.001 ppm to 100 ppm, or narrower alternative ranges from 0.1 ppm to 50 ppm, from 0.01 ppm to 40 ppm, from 0.05 ppm to 30 ppm, from 0.01 ppm to 25 ppm, or from 0.1 ppm to 30 ppm, or from 0.1 ppm to 25 ppm, or from 1 ppm to 30 ppm, or from 1 ppm to 25 ppm.

In some embodiments, the ingestible compositions disclosed herein, individually or in combination, may be provided in a flavoring concentrate formulation, e.g., suitable for subsequent processing to produce a ready-to-use (i.e., ready-to-serve) product. By “a flavoring concentrate formulation”, it is meant a formulation which should be reconstituted with one or more diluting medium to become a ready-to-use composition. The term “ready-to-use composition” is used herein interchangeably with “ingestible composition”, which denotes any substance that, either alone or together with another substance, can be taken by mouth whether intended for consumption or not. In one embodiment, the ready-to-use composition includes a composition that can be directly consumed by a human or animal. The flavoring concentrate formulation is typically used by mixing with or diluted by one or more diluting medium, e.g., any consumable or ingestible ingredient or product, to impart or modify one or more flavors to the diluting medium. Such a use process is often referred to as reconstitution. The reconstitution can be conducted in a household setting or an industrial setting. For example, a frozen fruit juice concentrate can be reconstituted with water or other aqueous medium by a consumer in a kitchen to obtain the ready-to-use fruit juice beverage. In another example, a soft drink syrup concentrate can be reconstituted with water or other aqueous medium by a manufacturer in large industrial scales to produce the ready-to-use soft drinks. Since the flavoring concentrate formulation has the flavoring agent or flavor modifying agent in a concentration higher than the ready-to-use composition, the flavoring concentrate formulation is typically not suitable for being consumed directly without reconstitution. There are many benefits of using and producing a flavoring concentrate formulation. For example, one benefit is the reduction in weight and volume for transportation as the flavoring concentrate formulation can be reconstituted at the time of usage by the addition of suitable solvent, solid or liquid.

The flavored products set forth according to any of the foregoing embodiments, also include, in certain embodiments, one or more additional flavor-modifying compounds, such as compounds that enhance sweetness (e.g., hesperetin, naringenin, glucosylated steviol glycosides, etc.), compounds that block bitterness, compounds that enhance umami, compounds that reduce sourness, compounds that enhance saltiness, compounds that enhance a cooling effect, or any combinations of the foregoing.

In certain embodiments of any aspects and embodiments set forth herein that refer to a sweetening or flavoring concentrate, the sweetening or flavoring concentrate is a non-naturally-occurring product, such as a composition specifically manufactured for the production of a flavored product, such as food or beverage product.

In one embodiment, the flavoring concentrate formulation comprises i) compounds as disclosed and described herein, individually or in combination; ii) a carrier; and iii) optionally at least one adjuvant. The term “carrier” denotes a usually inactive accessory substance, such as solvents, binders, or other inert medium, which is used in combination with the present compound and one or more optional adjuvants to form the formulation. For example, water or starch can be a carrier for a flavoring concentrate formulation. In some embodiments, the carrier is the same as the diluting medium for reconstituting the flavoring concentrate formulation; and in other embodiments, the carrier is different from the diluting medium. The term “carrier” as used herein includes, but is not limited to, ingestibly acceptable carrier.

The term “adjuvant” denotes an additive which supplements, stabilizes, maintains, or enhances the intended function or effectiveness of the active ingredient, such as the compound of the present invention. In one embodiment, the at least one adjuvant comprises one or more flavoring agents. The flavoring agent may be of any flavor known to one skilled in the art or consumers, such as the flavor of chocolate, coffee, tea, mocha, French vanilla, peanut butter, chai, or combinations thereof. In another embodiment, the at least one adjuvant comprises one or more sweeteners. The one or more sweeteners can be any of the sweeteners described in this application. In another embodiment, the at least one adjuvant comprises one or more ingredients selected from the group consisting of a emulsifier, a stabilizer, an antimicrobial preservative, an antioxidant, vitamins, minerals, fats, starches, protein concentrates and isolates, salts, and combinations thereof. Examples of emulsifiers, stabilizers, antimicrobial preservatives, antioxidants, vitamins, minerals, fats, starches, protein concentrates and isolates, and salts are described in U.S. Pat. No. 6,468,576, the content of which is hereby incorporated by reference in its entirety for all purposes.

In one embodiment, the present flavoring concentrate formulation can be in a form selected from the group consisting of liquid including solution and suspension, solid, foamy material, paste, gel, cream, and a combination thereof, such as a liquid containing certain amount of solid contents. In one embodiment, the flavoring concentrate formulation is in form of a liquid including aqueous-based and nonaqueous-based. In some embodiments, the present flavoring concentrate formulation can be carbonated or non-carbonated.

The flavoring concentrate formulation may further comprise a freezing point depressant, nucleating agent, or both as the at least one adjuvant. The freezing point depressant is an ingestibly acceptable compound or agent which can depress the freezing point of a liquid or solvent to which the compound or agent is added. That is, a liquid or solution containing the freezing point depressant has a lower freezing point than the liquid or solvent without the freezing point depressant. In addition to depress the onset freezing point, the freezing point depressant may also lower the water activity of the flavoring concentrate formulation. The examples of the freezing point depressant include, but are not limited to, carbohydrates, oils, ethyl alcohol, polyol, e.g., glycerol, and combinations thereof. The nucleating agent denotes an ingestibly acceptable compound or agent which is able to facilitate nucleation. The presence of nucleating agent in the flavoring concentrate formulation can improve the mouthfeel of the frozen Blushes of a frozen slush and to help maintain the physical properties and performance of the slush at freezing temperatures by increasing the number of desirable ice crystallization centers. Examples of nucleating agents include, but are not limited to, calcium silicate, calcium carbonate, titanium dioxide, and combinations thereof.

In one embodiment, the flavoring concentrate formulation is formulated to have a low water activity for extended shelf life. Water activity is the ratio of the vapor pressure of water in a formulation to the vapor pressure of pure water at the same temperature. In one embodiment, the flavoring concentrate formulation has a water activity of less than about 0.85. In another embodiment, the flavoring concentrate formulation has a water activity of less than about 0.80. In another embodiment, the flavoring concentrate formulation has a water activity of less than about 0.75.

In one embodiment, the flavoring concentrate formulation has the present compound in a concentration that is at least 2 times of the concentration of the compound in a ready-to-use composition. In one embodiment, the flavoring concentrate formulation has the present compound in a concentration that is at least 5 times of the concentration of the compound in a ready-to-use composition. In one embodiment, the flavoring concentrate formulation has the present compound in a concentration that is at least 10 times of the concentration of the compound in a ready-to-use composition. In one embodiment, the flavoring concentrate formulation has the present compound in a concentration that is at least 15 times of the concentration of the compound in a ready-to-use composition. In one embodiment, the flavoring concentrate formulation has the present compound in a concentration that is at least 20 times of the concentration of the compound in a ready-to-use composition. In one embodiment, the flavoring concentrate formulation has the present compound in a concentration that is at least 30 times of the concentration of the compound in a ready-to-use composition. In one embodiment, the flavoring concentrate formulation has the present compound in a concentration that is at least 40 times of the concentration of the compound in a ready-to-use composition. In one embodiment, the flavoring concentrate formulation has the present compound in a concentration that is at least 50 times of the concentration of the compound in a ready-to-use composition. In one embodiment, the flavoring concentrate formulation has the present compound in a concentration that is at least 60 times of the concentration of the compound in a ready-to-use composition. In one embodiment, the flavoring concentrate formulation has the present compound in a concentration that is up to 100 times of the concentration of the compound in a ready-to-use composition.

In some embodiments, the flavorings may be used in many distinct physical forms well-known in the art to provide an initial burst of flavor and/or a prolonged sensation of flavor. Without being limited thereto, such physical forms include free forms, such as spray dried, powdered, beaded forms, encapsulated forms, and mixtures thereof.

In some embodiments, the ingestible composition comprises a flavor-modifying compound according to any of the embodiments set forth above and a bulking agent. Suitable bulking agents include, but are not limited to maltodextrin (10 DE, 18 DE, or 5 DE), corn syrup solids (20 or 36 DE), sucrose, fructose, glucose, invert sugar, sorbitol, xylose, ribulose, mannose, xylitol, mannitol, galactitol, erythritol, maltitol, lactitol, isomalt, maltose, tagatose, lactose, inulin, glycerol, propylene glycol, polyols, polydextrose, fructooligosaccharides, cellulose and cellulose derivatives, and the like, and mixtures thereof. Additionally, granulated sugar (sucrose) or other caloric sweeteners such as crystalline fructose, other carbohydrates, or sugar alcohols can be used as a bulking agent due to their provision of good content uniformity without the addition of significant calories.

In one embodiment, the at least one bulking agent may be a bulking agent described in U.S. Pat. No. 8,993,027.

In one embodiment, the at least one bulking agent may be a bulking agent described in U.S. Pat. No. 6,607,771.

In one embodiment, the at least one bulking agent may be a bulking agent described in U.S. Pat. No. 6,932,982.

In some embodiments, the tabletop sweetener composition may further comprise at least one anti-caking agent. As used herein the phrase “anti-caking agent” and “flow agent” refer to any composition which prevents, reduces, inhibits, or suppresses the at least one sweetener from attaching, binding, or contacting to another sweetener molecule. Alternatively, anti-caking agent may refer to any composition which assists in content uniformity and uniform dissolution. Non-limiting examples of anti-caking agents include cream of tartar, calcium silicate, silicon dioxide, microcrystalline cellulose (Avicel, FMC BioPolymer, Philadelphia, Pa.), and tricalcium phosphate. In one embodiment, the anti-caking agents are present in the tabletop sweetener composition in an amount from about 0.001 to about 3% by weight of the tabletop sweetener composition.

In some embodiments, the sweetener compositions of any of the preceding aspects and embodiments thereof are encapsulated using typical means for encapsulating flavor or fragrance compounds. Non-limiting examples of such technology are set forth in U.S. Patent Application Publication Nos. 2016/0235102, 2019/0082727, 2018/0369777, 2018/0103667, 2016/0346752, 2015/0164117, 2014/0056836, 2012/0027866, 2010/0172945, and 2007/0128234, as well as U.S. Pat. Nos. 7,488,503, 6,416,799, 5,897,897, 5,786,017, 5,603,971, 4,689,235, 4,610,890, 3,704,137, 3,041,180, and 2,809,895. All of the preceding patent publications and patents are hereby incorporated by reference as though set forth herein in their entireties.

### Non-Animal Protein Materials and Products Made Therefrom

Products intended to replace or substitute meat or dairy products often rely on various non-animal-based materials, such as starches and proteins derived from plants, algae, fungi, or combinations thereof, to simulate the texture and flavor of meat or dairy. Non-limiting examples of non-animal-based proteins are plant proteins, such as pea protein, soy protein, almond protein, cashew protein, canola (rapeseed) protein, chickpea protein, fava protein, sunflower protein, wheat protein, oat protein, barley protein, potato protein, and combinations thereof. Due to compositional differences between such plant-based materials and animal-derived materials, such as a lack of glutamate-containing proteins and glutathione, these products can lack the umami or kokumi taste that consumers traditionally associate with meat or dairy products, or may have bitter tastes that animal proteins lack.

Thus, in certain aspects, the disclosure provides a flavored product comprising a plant-based material (such as a plant-based starch, a plant-based protein, or a combination thereof) and zinc salts, according to any of the embodiments set forth above. In some further embodiments, the flavored product can include any features of combination of features set forth above for ingestible compositions that contain the zinc salts. In some embodiments, the flavored product is a beverage, such as soy milk, almond milk, rice milk, oat milk, a protein drink, a meal-replacement drink, or other like product. In some other embodiments, the flavored product is a meat-replacement product, such as a plant-based chicken product (such as a plant-based chicken nugget), a plant-based beef product (such as a plant-based burger), and the like. In some other embodiments, the flavored product is a protein powder, a meal-replacement powder, a plant-based creamer for coffee or tea, and the like. In certain further embodiments, any such products contain additional ingredients, and have additional features, as are typically used in the preparation and/or manufacture of such products. For example, the flavor-modifying compounds may be combined with other flavors and taste modifiers, and may even be encapsulated in certain materials, according to known technologies in the relevant art. Suitable concentrations of the flavor-modifying compounds are set forth above.

In some embodiments, the flavored products comprise one or more plant-based proteins, which impart a bitter taste that is at least partially reduced by the use of the flavor-modifying compounds in the product. Such plant-based proteins include, but are not limited to, pea protein, soy protein, almond protein, cashew protein, canola (rapeseed) protein, chickpea protein, fava protein, sunflower protein, wheat protein, oat protein, barley protein, potato protein, and combinations thereof.

In some alternative embodiments analogous to the above embodiments, algal or fungal proteins or starches are used instead. In some embodiments, these flavored products also include fiber to provide texture to the product. Fibers suitable for use include, but are not limited to, psyllium fiber, pea fiber, potato fiber, curdlan, soluble corn fiber (dextran and/or maltodextrin), citrus fiber, and combinations thereof. In such products, the flavor-modifying compounds can be introduced in any suitable way. In some embodiments, the flavor-modifying compounds are incorporated into a flavoring emulsion, such as a water-in-oil emulsion, along with other flavor-imparting ingredients.

### Non-Meat Protein Materials and Products Made Therefrom

Certain non-meat animal proteins, such as dairy proteins and proteins from bone broth, are commonly used in food products, and are also sold as the primary ingredient in certain protein powders. Such proteins can impart bitter flavors that consumers may not desire. This is especially true for protein isolates, such as protein isolates of whey protein, collagen protein, casein proteins, and the like. Thus, the present disclosure provides ingestible compositions that include non-meat animal proteins and the flavor-modifying compounds. The flavor-modifying compounds can be present in any suitable combination, according to the embodiments set forth in the preceding sections of the present disclosure. In some embodiments, the non-meat animal protein is a bone protein, such as a collagen protein derived from the bones of an animal, such as a cow, pig, donkey, horse, chicken, duck, goat, goose, rabbit, lamb, sheep, buffalo, ostrich, camel, and the like. In some embodiments, the non-meat animal protein is a milk protein, such as a whey protein, a casein protein, or any combination thereof. The milk can be the milk of any suitable animal, such as a cow, donkey, horse, sheep, buffalo, camel, and the like.

The flavor-modifying compounds can also be included in certain food or beverage products that include animal milk or materials derived from animal milk. Such products include cheeses, cheese spreads, yogurt, kefir, milk, processed dairy products, cottage cheese, sour cream, butter, and the like.

### Blocking Bitterness in Pharmaceutical APIs

Many drug compounds impart a bitter taste, which therefore limits the ways in which they can be formulated and administered. Therefore, in certain aspects, the disclosure provides a pharmaceutical composition comprising a bitter-tasting pharmaceutical active ingredient, and the flavor-modifying compounds. Such pharmaceutical compositions can be in any suitable form for oral administration, such as tablets, lozenges, capsules, powders, liquid solutions, liquid suspensions, and the like. Such pharmaceutical compositions can include any suitable pharmaceutical excipients, binders, and the like, such as those set forth in Remington's Pharmaceutical Sciences. In some embodiments, the bitter-tasting pharmaceutical active ingredient is an ion channel inhibitor, such as a proton channel inhibitor. Other examples of bitter-tasting APIs whose bitterness is reduced by flavor-modifying compounds include, but are not limited to, atropine, brinzolamide, chloramphenicol, chloroquine, clindamycin, dexamethasone, digoxin, diltiazem, diphenhydramine, docusate, dorzolamide, doxepin, doxylamine, enalapril, erythromycin, esomeprazole, famotidine, gabapentin, ginkgolide A, guaifenesin, L-histidine, lomefloxacin, methylprednisolone, ofloxacin, oleuropein, oxyphenonium, pirenzepine, prednisone, ranitidine, trapidil, trimethoprim, and cetirizine.

### Use in Oral Care Products

Oral care products often contain ingredients that impart astringent or bitter off tastes. Such ingredients include menthol, menthol analogues, mint extracts, sodium bicarbonate, alkali metal salts of peroxymonosulfate (potassium peroxymonosulfate), cetylpyridinium chloride, lauramidopropyl betaine, cocamidopropyl betaine, arginine, hydrogen peroxide, chlorhexidine gluconate, potassium nitrate, pentasodium triphosphate, tetrasodium pyrophosphate, stannous fluoride, thymol, methyl salicylate, eucalyptol, or any combination thereof. Suitable oral care products include toothpaste, mouthwashes, whitening agents, dentifrices, and the like. Such oral care products may comprise flavor-modifying compounds to block or mask the bitterness of such compounds.

### EXAMPLES

To further illustrate this invention, the following examples are included. The examples should not, of course, be construed as specifically limiting the invention. Variations of these examples within the scope of the claims are within the purview of one skilled in the art and are considered to fall within the scope of the invention as described, and claimed herein. The reader will recognize that the skilled artisan, armed with the present disclosure, and skill in the art is able to prepare and use the invention without exhaustive examples.

### Example 1: Preparation of (2,3-trans)-5-hydroxy-2-(3-hydroxy-4-methoxyphenyl)-4-oxochroman-3-yl acetate (101)

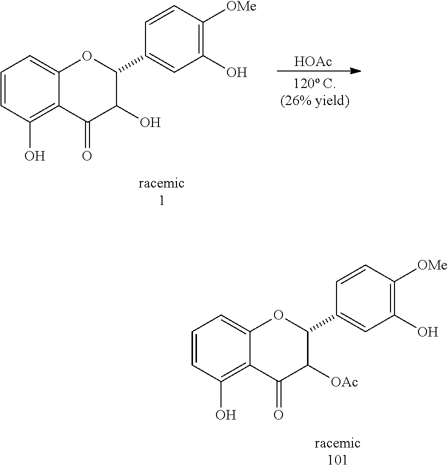 [SMILES: COc1ccc([C@H]2Oc3cccc(O)c3C(=O)C2O)cc1O ; COc1ccc([C@H]2Oc3cccc(O)c3C(=O)C2OC(C)=O)cc1O ; "HOAc"]

A 250 mL pressure flask, equipped with stir bar, was charged with a solution of flavanonol 1 (example 1a, 1.0 g, 3.3 mmol) in AcOH (70 mL). The solution was degassed by vigorously bubbling N₂ through the solution for 15 minutes. The headspace was sparged with N₂ and the sealed solution was heated at 120° C. for 60 hours. The solvent was removed under reduced pressure and the residue was partitioned between EtOAc (100 mL) and brine (100 mL), aqueous layer was extracted (2×100 mL EtOAc) and combined organics were dried over MgSO4, filtered and concentrated. The crude residue was purified by preparative RP HPLC (10→90% MeCN/H₂O) to provide 290 mg (25% yield) of 101 as a pale yellow oil. ¹H NMR (400 MHz, MeOD) δ 7.46 (t, J=8.3 Hz, 1H), 6.98 (s, 1H), 6.95 (s, 2H), 6.55 (d, J=8.4 Hz, 1H), 6.52 (d, J=8.3 Hz, 1H), 5.87 (d, J=12.0 Hz, 1H), 5.38 (d, J=12.0 Hz, 1H), 3.88 (s, 3H), 1.99 (s, 3H); MS (ESI) calculated for C₁₈H₁₇O₇ [M+H]⁺345.1, found 345.0.

### Example 1a: Preparation of (2,3-trans)-3,5-dihydroxy-2-(3-hydroxy-4-methoxyphenyl)-chroman-4-one (1)

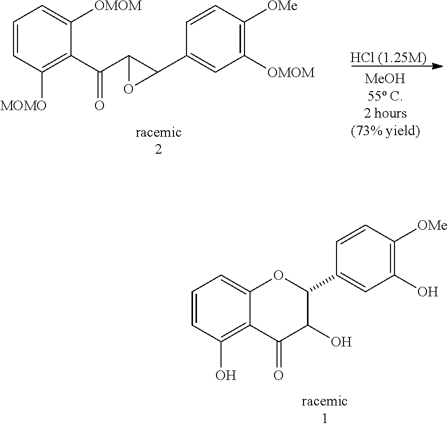

A 100 mL round-bottom flask, equipped with stir bar, condenser (air-cooled) rubber septa, was charged with the epoxide 2 (example 1b, 816 mg, 188 mmol), followed by dry methanolic HCl (1.25 M, 20 mL) under N₂. The reaction mixture was heated at 55° C. for 2 hours, after which time LCMS analysis indicated consumption of the starting material. Solvent was removed under reduced pressure and the residue was redissolved in MeOH (150 mL) and the solvent was removed under reduced pressure. The crude residue was purified by preparative RP HPLC (10→90% MeCN/H₂O) to provide 400 mg (71% yield) of flavanonol 1 as white solid. ¹H NMR (400 MHz, MeOD) δ 7.42 (td, J=8.3, 0.8 Hz, 1H), 7.02 (d, J=1.8 Hz, 1H), 7.00-6.90 (m, 2H), 6.51 (dd, J=8.3, 0.9 Hz, 1H), 6.47 (dd, J=8.3, 0.9 Hz, 1H), 5.04 (d, J=11.8 Hz, 1H), 4.62 (dd, J=11.8, 0.8 Hz, 1H), 3.88 (d, J=0.7 Hz, 3H); ¹³C NMR (101 MHz, MeOD) δ 199.8, 161.7, 161.5, 148.2, 146.2, 138.2, 129.6, 119.4, 114.2, 110.9, 108.8, 107.2, 106.4, 83.6, 72.8, 55.0; MS (ESI) calculated for C₁₆H₁₅O₆ [M+H]⁺303.0, found 303.1

### Example 1b: Preparation of (E)-1-(2,6-bis(methoxymethoxy)phenyl)-3-(4-methoxy-3-(methoxymethoxy)phenyl)prop-2-en-1-one (2)

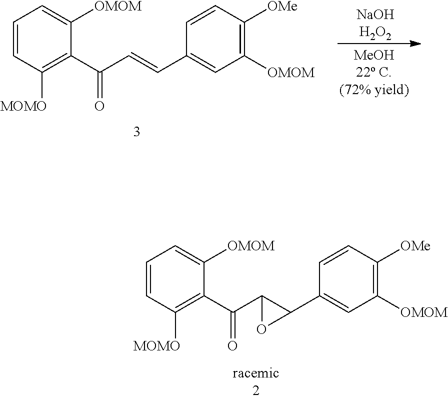

To an emulsion of the chalcone 3 (example 1c, 1.8 g, 4.4 mmol) in methanol (120 mL) was added NaOH (530 mg, 13 mmol) at 22° C. in one portion followed by a dropwise addition of aqueous solution of hydrogen peroxide (30% wt, 3.6 mL, 35 mmol). The reaction mixture was left stirred for 18 hours, after which time LCMS analysis indicated consumption of the starting material. The reaction was quenched with a saturated aqueous NH₄Cl solution (120 mL) and extracted with EtOAc (3×100 mL). The organics were combined, dried over MgSO₄, filtered and concentrated under reduced pressure. The crude residue was purified by silica gel chromatography (0→60% EtOAc/hexanes) to provide 1.36 g (72% yield) of chalcone epoxide 2 as a light yellow oil. ¹H NMR (400 MHz, CDCl₃) δ 7.28 (t, J=8.4 Hz, 1H), 7.06 (d, J=2.1 Hz, 1H), 6.95 (dd, J=8.4, 2.1 Hz, 1H), 6.86 (d, J=8.4 Hz, 1H), 6.82 (d, J=8.4 Hz, 2H), 5.27-5.19 (m, 2H), 5.18-5.14 (m, 4H), 3.97 (d, J=1.8 Hz, 1H), 3.89 (d, J=1.8 Hz, 1H), 3.87 (s, 3H), 3.49 (s, 3H), 3.42 (s, 6H); MS (ESI) calculated for C₂₂H₂₇O₉ [M+H]⁺ 435.2, found 435.2.

### Example 1c: Preparation of (E)-1-(2,6-bis(methoxymethoxy)phenyl)-3-(4-methoxy-3-(methoxymethoxy)phenyl)prop-2-en-1-one (3)

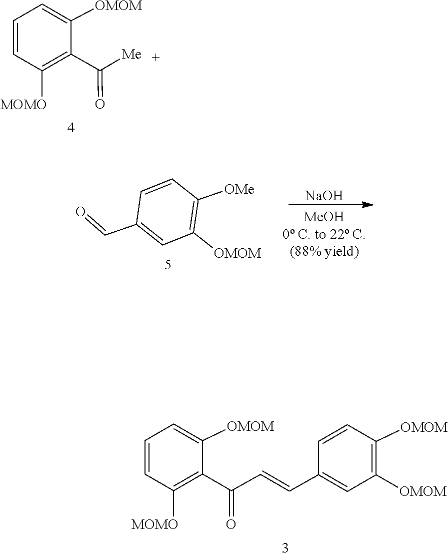

To a solution of 4 (example 1d, 3.50 g, 14.6 mmol) in methanol (40 mL) was added an aqueous NaOH (30% wt, 20 mL, 150 mmol) at 0° C. After stirring for 30 minutes, a solution of 5 (example 1e, 3.44 g, 17.5 mmol) in MeOH (35 mL) was added dropwise. The reaction mixture was left stirred for 20 hours (the ice bath was allowed to melt), after which time LCMS analysis indicated consumption of the starting materials. The mixture was diluted with EtOAc (75 mL) and washed with saturated aqueous NH₄Cl solution (2×75 mL). The aqueous layers were combined and extracted with (2×75 mL EtOAc). The organic layers were combined, dried over MgSO₄, filtered, and concentrated under reduced pressure. The crude residue was purified by silica gel chromatography (0→100% EtOAc/hexanes) to provide 5.70 g (93% yield) of chalcone 3 as a yellow solid. ¹H NMR (400 MHz, CDCl₃) δ 7.36 (d, J=2.1 Hz, 1H), 7.29 (t, J=8.4 Hz, 1H), 7.23 (d, J=16.1 Hz, 1H), 7.16 (dd, J=8.6, 2.0 Hz, 1H), 6.91-6.83 (m, 4H), 5.23 (s, 2H), 5.14 (s, 4H), 3.91 (s, 3H), 3.51 (s, 3H), 3.39 (s, 6H); MS (ESI) calculated for C₂₂H₂₇O₈ [M+H]⁺ 419.2, found 419.2.

### Example 1d: Preparation of 1-(2,6-bis(methoxymethoxy)phenyl)ethan-1-one (4)

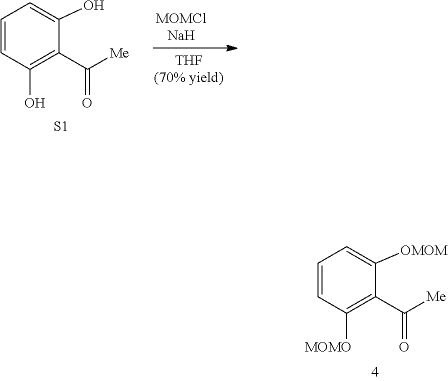

A 500 mL oven-dried round-bottom flask, equipped with stir bar and rubber septa, was charged with NaH (4.0 g, 60% dispersion mineral oil, 100 mmol), followed by anhydrous THF (80 mL) under N₂. The suspension was cooled to 0° C. in an ice bath, and a solution of 2′,6′-dihydroxyacetophenone (S1, 5.0 g, 33 mmol) in THF (100 mL) was added dropwise over 15 minutes (flask was vented with 22G needle to avoid overpressurization). After stirring for 20 minutes (cessation of gas evolution) at 22° C., the solution was cooled back to 0° C. and MOMCl (7.5 mL, 99 mmol) was added dropwise to the suspension at 0° C. over 15 minutes. The reaction was left stirred for 12 hours (the ice bath was allowed to melt), after which time LCMS analysis indicated consumption of the starting material. Ice-water (150 mL) was added slowly, followed by extraction with EtOAc (3×150 mL). The combined organic layers were dried over MgSO₄, filtered, and concentrated under reduced pressure. The crude residue was purified by silica gel chromatography (0→40% EtOAc/hexanes) to provide 5.6 g (70% yield) of acetophenone 4 as a colorless oil. ¹H NMR (400 MHz, CDCl₃) δ 7.22 (t, J=8.4 Hz, 1H), 6.80 (d, J=8.4 Hz, 2H), 5.16 (s, 4H), 3.46 (s, 6H), 2.52 (s, 3H); MS (ESI) calculated for C₁₁H₁₃O₄ [M−OMe−]⁺ 209.1, found 209.0.

### Example 1e: Preparation of 4-methoxy-3-(methoxymethoxy)benzaldehyde (5)

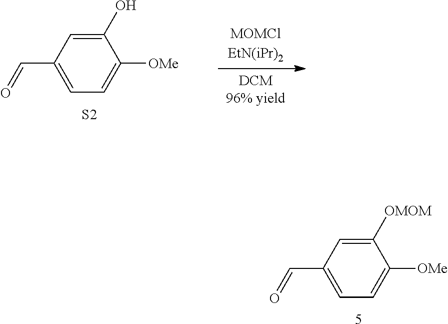

A 1L oven-dried round-bottom flask, equipped with stir bar and rubber septa, was charged with isovanillin (S2, 10 g, 66 mmol), followed by dry DCM (400 mL) under N₂. The mixture was cooled to 0° C. in an ice bath, and a DIPEA (16 mL, 92 mmol) was added slowly. After stirring for 20 min, MOMCl (6.0 mL, 80 mmol) was added dropwise at 0° C. over 15 minutes. The reaction was left stirred for 18 hours (the ice bath was allowed to melt), after which time LCMS analysis indicated consumption of the starting materials. Water (300 mL) was added, followed by extraction with DCM (3×150 mL). The combined organic layers were dried over MgSO₄, filtered, and concentrated under reduced pressure. The crude residue was purified by silica gel chromatography (0→50% EtOAc/hexanes) to provide compound 5 (12.7 g, 98% yield) as a white solid. ¹H NMR (400 MHz, CDCl₃) δ 9.86 (s, 1H), 7.67 (d, J=2.0 Hz, 1H), 7.55 (dd, J=8.3, 1.9 Hz, 1H), 7.02 (d, J=8.3 Hz, 1H), 5.29 (s, 2H), 3.97 (s, 3H), 3.53 (s, 3H); MS (ESI) calculated for C₁₀H₁₃O₄ [M+H]⁺ 197.1, found 197.0.

### Example 2: Preparation of (2,3-trans)-5-hydroxy-2-(3-hydroxy-4-methoxyphenyl)-4-oxochroman-3-yl propionate (102)

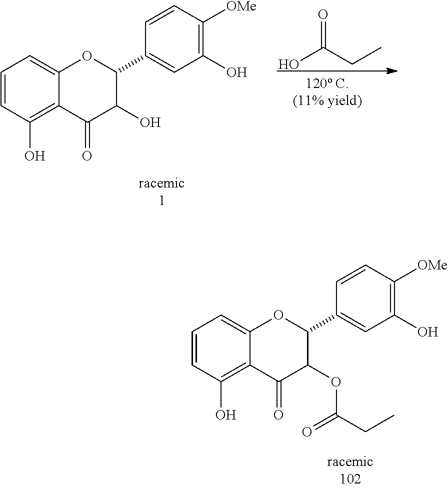

Prepared as in example 1 from 1 (example 1a) and propionic acid to give compound 102 (2.3 mg, 11% yield) of clear film. ¹H NMR (500 MHz, MeOD) δ 7.49 (t, J=8.3 Hz, 1H), 7.00 (q, J=1.6 Hz, 1H), 6.98 (d, J=1.4 Hz, 2H), 6.58 (dd, J=8.4, 1.0 Hz, 1H), 6.55 (dd, J=8.3, 1.0 Hz, 1H), 5.90 (d, J=12.1 Hz, 1H), 5.41 (d, J=12.1 Hz, 1H), 3.90 (s, 3H), 2.36 (dq, J=16.6, 7.6 Hz, 1H), 2.27 (dq, J=16.5, 7.5 Hz, 1H), 1.03 (t, J=7.6 Hz, 3H).

### Example 3: Preparation of (2,3-trans)-7-hydroxy-2-(3-hydroxy-4-methoxyphenyl)-4-oxochroman-3-yl acetate (103)

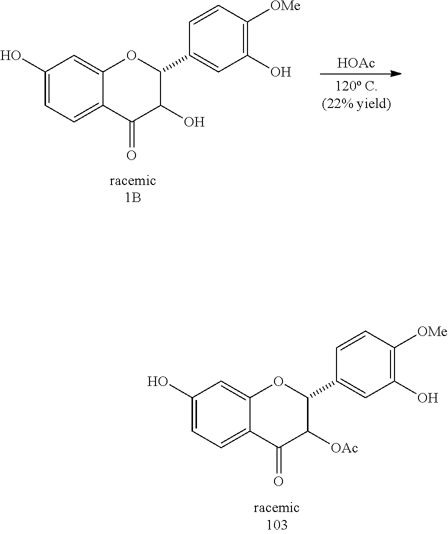

A 250 mL pressure flask, equipped with stir bar, was charged with a solution of flavanonol 1B (example 3a, 1.11 g, 3.67 mmol) in AcOH (85 mL). The solution was degassed by vigorously bubbling N₂ through the solution for 15 minutes. The headspace was sparged with N₂ and the sealed solution was heated at 120° C. for 60 hours. The solvent was removed under reduced pressure and the residue was partitioned between EtOAc (100 mL) and brine (100 mL), aqueous layer was extracted (2×100 mL EtOAc) and combined organics were dried over MgSO₄, filtered and concentrated. The crude residue was purified by preparative RP HPLC (25→45% MeCN/H₂O) to provide 260 mg (22% yield) of 103 as a pale yellow oil. ¹H NMR (400 MHz, MeOD) δ 7.72 (d, J=8.7 Hz, 1H), 6.97 (t, J=1.3 Hz, 1H), 6.94 (d, J=1.3 Hz, 2H), 6.55 (dd, J=8.7, 2.2 Hz, 1H), 6.36 (d, J=2.2 Hz, 1H), 5.72 (d, J=12.0 Hz, 1H), 5.29 (d, J=12.0 Hz, 1H), 3.87 (s, 3H), 1.96 (s, 3H); MS (ESI) calculated for C₁₈H₁₇O₇ [M+H]⁺ 345.1, found 345.0.

### Example 3a: Preparation of (2,3-trans)-3,7-dihydroxy-2-(3-hydroxy-4-methoxyphenyl)-chroman-4-one-methanol (1B)

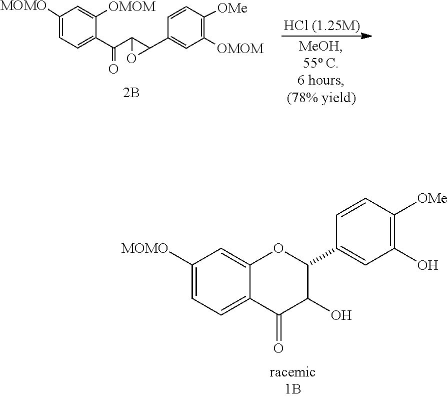

A 100 mL round-bottom flask, equipped with stir bar and an air condenser was charged with 2B (example 3b, 2.3 g, 510 μmol), followed by dry methanolic HCl (1.25 M, 65 mL) under N₂. The reaction mixture was heated at 55° C. for 6 hours, after which time LCMS analysis indicated consumption of the starting material. Solvent was removed under reduced pressure and the residue was redissolved in MeOH (50 mL) and the solvent was removed under reduced pressure. The crude residue was purified by preparative RP HPLC (10→90% MeCN/H₂O) to provide 1.21 g (78% yield) of flavanonol 1B as a clear oil. ¹H NMR (400 MHz, MeOD) δ 7.72 (d, J=8.7 Hz, 1H), 7.01 (d, J=1.8 Hz, 1H), 7.00-6.88 (m, 2H), 6.53 (dd, J=8.7, 2.2 Hz, 1H), 6.33 (d, J=2.2 Hz, 1H), 4.97 (d, J=11.8 Hz, 1H), 4.48 (d, J=11.8 Hz, 1H), 3.88 (s, 3H); MS (ESI) calculated for C₁₆H₁₅P₆ [M+H]⁺ 303.0, found 303.1.

### Example 3b: Preparation of (E)-1-(2,6-bis(methoxymethoxy)phenyl)-3-(4-methoxy-3-(methoxymethoxy)phenyl)prop-2-en-1-one (2B)

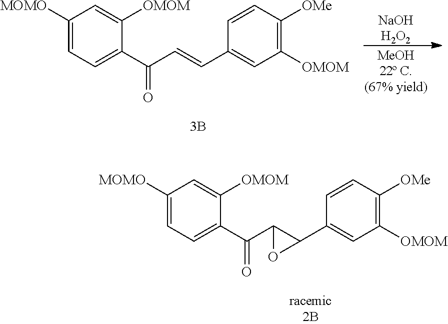

To an emulsion of chalcone 3B (example 3c, 3.3 g, 7.9 mmol) in methanol (200 mL) was added NaOH (950 mg, 24 mmol) at 22° C. in one portion followed by a dropwise addition of aqueous solution of hydrogen peroxide (30% wt. aqueous solution, 6.4 mL, 63 mmol). The reaction mixture was left stirred for 15 hours, after which time LCMS analysis indicated consumption of the starting material. The solvent was partially removed under vacuum and the mixture was diluted with MBTE (200 mL) washed with saturated aqueous NH₄Cl solution (200 mL) and extracted with MBTE (2×150 mL). The organics were combined, dried over MgSO₄, filtered and concentrated under reduced pressure. The crude residue was purified by silica gel chromatography (0→60% EtOAc/hexanes) to provide 2.3 g (67% yield) of chalcone epoxide 2B as a light yellow oil. ¹H NMR (400 MHz, CDCl₃) δ 7.84 (d, J=8.6 Hz, 1H), 7.12 (d, J=2.1 Hz, 1H), 7.02 (dd, J=8.3, 2.1 Hz, 1H), 6.90 (d, J=8.3 Hz, 1H), 6.81-6.74 (m, 2H), 5.26-5.19 (m, 4H), 4.96 (d, J=6.9 Hz, 1H), 4.87 (d, J=6.9 Hz, 1H), 4.32 (d, J=2.0 Hz, 1H), 3.92 (d, J=2.0 Hz, 1H), 3.90 (s, 3H), 3.48 (d, J=9.3 Hz, 6H), 3.15 (s, 3H); MS (ESI) calculated for C₂₂H₂₇O₉ [M+H]⁺ 435.2, found 435.2.

### Example 3c: Preparation of (E)-1-(2,6-bis(methoxymethoxy)phenyl)-3-(4-methoxy-3-(methoxymethoxy)phenyl)prop-2-en-1-one (3B)

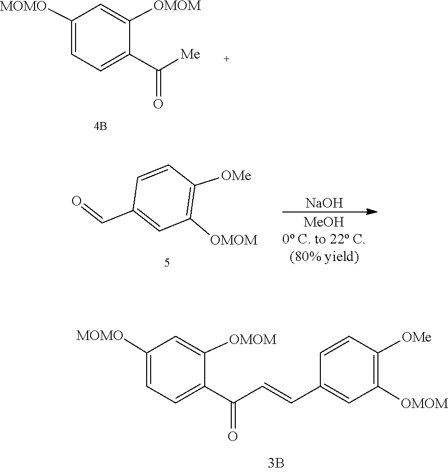

To a solution of 4B (example 3d, 2.0 g, 8.3 mmol) in methanol (30 mL) was added an aqueous NaOH (30% wt, 6 mL, 42 mmol) at 0° C. After stirring for 30 minutes, a solution of MOM-isovanillin 5 (example 1e, 1.8 g, 9.2 mmol) in MeOH (15 mL) was added dropwise. The reaction mixture was left stirred for 45 hours (the ice bath was allowed to melt), after which time LCMS analysis indicated consumption of the starting materials. The mixture was diluted with Et₂O (50 mL) and washed with saturated aqueous NH₄Cl solution (2×30 mL). The aqueous layers were combined and extracted with (2×40 mL Et₂O). The organic layers were combined, dried over MgSO₄, filtered, and concentrated under reduced pressure. The crude residue was purified by silica gel chromatography (0→60% EtOAc/hexanes) to provide 2.8 g (80% yield) of chalcone 3B as a yellow oil. ¹H NMR (400 MHz, CDCl₃) δ 7.66 (d, J=8.7 Hz, 1H), 7.59 (d, J=15.8 Hz, 1H), 7.46 (d. J=2.1 Hz, 1H), 7.35 (d, J=15.7 Hz, 1H), 7.22 (dd, J=8.5, 2.1 Hz, 1H), 6.90 (d, J=8.4 Hz, 1H), 6.86 (d, J=2.2 Hz, 1H), 6.77 (dd, J=8.6, 2.3 Hz, 1H), 5.25 (d, J=0.5 Hz, 4H), 5.22 (s, 2H), 3.92 (s, 3H), 3.52 (s, 3H), 3.51 (s, 3H), 3.50 (s, 3H); MS (ESI) calculated for C₂₂H₂₇O₈ [M+H]⁺ 419.2, found 419.2.

### Example 3d: Preparation of 1-(2,4-bis(methoxymethoxy)phenyl)ethan-1-one (4B)

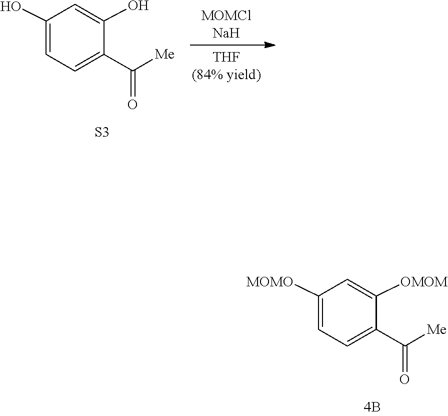

A 500 mL oven-dried round-bottom flask, equipped with stir bar and rubber septa, was charged with NaH (4.10 g, 60% dispersion mineral oil, 103 mmol), followed by dry DCM (200 mL) under N₂. The suspension was cooled to 0° C. in an ice bath, and a solution of 2′, 4′,-dihydroxyacetophenone S3 (4.46 g, 29.3 mmol) in DMF (10 mL) was added dropwise over 15 minutes (flask was vented with 22G needle to avoid overpressurization). After stirring for 25 min (cessation of gas evolution), MOMCl (5.12 mL, 67.4 mmol) was added dropwise to the suspension at 0° C. over 5 minutes. The reaction was left stirred for 19 hours (the ice bath was allowed to melt), after which time LCMS analysis indicated consumption of the starting material. Water (40 mL) was added, followed by extraction with DCM (3×100 mL). The combined organic layers were dried over MgSO₄, filtered, and concentrated under reduced pressure. The crude residue was purified by silica gel chromatography (0-≥40% EtOAc/hexanes) to provide 5.9 g (84% yield) of acetophenone 4B as a clear oil. ¹H NMR (400 MHz, CDCl₃) δ 7.78 (d, J=8.8 Hz, 1H), 6.82 (d, J=2.3 Hz, 1H), 6.72 (dd, J=8.7, 2.3 Hz, 1H), 5.27 (s, 2H), 5.20 (s, 2H), 3.52 (s, 3H), 3.48 (s, 3H), 2.60 (s, 3H); MS (ESI) calculated for C₁₁H₁₃O₄ [M−OMe]⁺ 209.0, found 209.0.

### Example 4: Preparation of (2,3-trans)-7-hydroxy-2-(3-hydroxy-4-methoxyphenyl)-4-oxochroman-3-yl propionate (104)

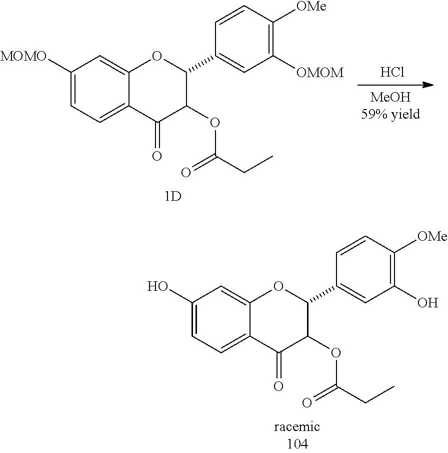

To a solution of 1D (example 4a, 22 mg, 49 μmol) in MeOH (5 mL) was added concentrated HCl (1 mL). The resulting solution was left starred at 22° C. for 60 minutes, after which time LCMS analysis indicated consumption of the starting material. The solvent was removed under reduced pressure and the residue redissolved again in MeOH (5 mL) and the solvent was removed under reduced pressure. The crude residue was purified by preparative RP HPLC (10→90% MeCN/H₂O) to provide 104 (10 mg, 59% yield) as a clear oil. ¹H NMR (500 MHz, MeOD) δ 7.75 (d, J=8.8 Hz, 1H), 6.99 (d, J=1.4 Hz, 1H), 6.97 (d, J=1.2 Hz, 2H), 6.59 (dd, J=8.7, 2.2 Hz, 1H), 6.40 (d, J=2.2 Hz, 1H), 5.75 (d, J=12.0 Hz, 1H), 5.33 (d, J=12.0 Hz, 1H), 3.90 (s, 3H), 2.41-2.28 (m, 1H), 2.24 (dt, J=16.5, 7.5 Hz, 1H), 1.01 (t, J=7.5 Hz, 3H); MS (ESI) calculated for C₁₉H₁₉O₆ [M+H]⁺ 359.1, found 359.0.

### Example 4a: Preparation of (2,3-trans)-2-(4-methoxy-3-(methoxymethoxy)phenyl)-7-(methoxymethoxy)-4-oxochroman-3-yl propionate (1D)

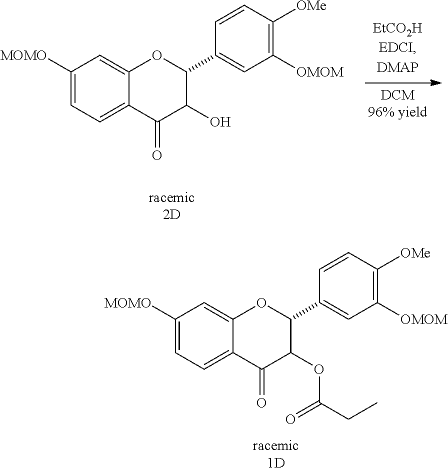

To a solution of 2D (example 4b, 20 mg, 51 μM) in DCM (3 mL) was added DMAP (13 mg, 110 μM), EDCl (21 mg, 110 μM) followed by a solution of propionic acid (7.6 mg, 100 μM) in DCM (1 mL). The resulting solution was left starred at 22° C. for 18 hours, after which time LCMS analysis indicated consumption of the starting material. The reaction mixture was directly loaded onto silica column and purified by silica gel chromatography (0 →50% EtOAc/hexanes) to provide 22 mg (96% yield) of ester 1D as a clear oil. ¹H NMR (500 MHz, CDCl₃) δ 7.71 (d, J=8.8 Hz, 1H), 7.17 (d, J=2.1 Hz, 1H), 7.08 (dd, J=8.4, 2.1 Hz, 1H), 6.94 (d, J=8.4 Hz, 1H), 6.70 (dd, J=8.8, 2.3 Hz, 1H), 6.59 (d, J=2.2 Hz, 1H), 5.72 (d, J=12.2 Hz, 1H), 5.31 (d, J=12.2 Hz, 1H), 5.17 (s, 2H), 5.10 (d, J=6.5 Hz, 1H), 5.06 (d, J=6.6 Hz, 1H), 4.75 (s, 5H), 3.77 (s, 3H), 3.39 (s, 3H), 3.36 (s, 3H), 2.23 (dq, J=16.4, 7.5 Hz, 1H), 2.13 (dq, J=16.5, 7.6 Hz, 1H), 0.88 (t, J=7.6 Hz, 3H).

### Example 4b. Preparation of (2,3-trans)-3-hydroxy-2-(4-methoxy-3-(methoxymethoxy)-phenyl)-7-(methoxymethoxy)chroman-4-one (2D)

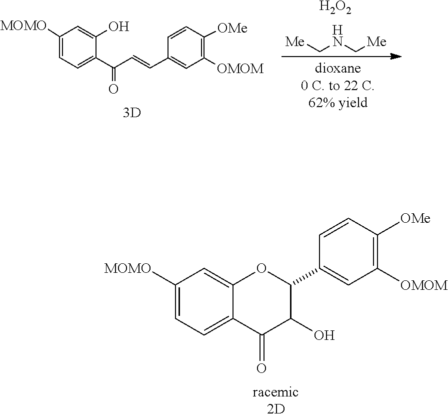

To a solution of 3D (example 4c, 800 mg, 2 mmol) in dioxane (25 mL) was added Et₂NH (1.1 mL, 11 mmol) under N₂ followed by slow addition of H₂O₂ (30% wt, 6.6 mL, 64 mmol) over 15 minutes. The reaction mixture was left stirred vigorously for 18 hours, after which time LCMS analysis indicated consumption of the starting material. The solution was cooled to 0° C. on an ice bath and a solution of Na₂S₂O₃·5H₂O (50 g, 0.28 mol) in H₂O (~150 mL) was added slowly (caution exotherm!) over 30 minutes to quench the excess of hydrogen peroxide, followed by extraction with EtOAc (3×100 mL). The organic layers were combined, dried over MgSO₄, filtered, and concentrated under reduced pressure. The crude residue was purified by silica gel chromatography (25→40% EtOAc/hexanes) to provide compound 2D 513 mg (62% yield) as a white solid. ¹H NMR (400 MHz, CDCl₃) δ 7.86 (d, J=8.8 Hz, 1H), 7.39 (d. J=2.1 Hz, 1H), 7.19 (dd, J=8.4, 2.1 Hz, 1H), 6.97 (d, J=8.4 Hz, 1H), 6.76 (dd, J=8.8, 2.3 Hz, 1H), 6.68 (d, J=2.3 Hz, 1H), 5.37-5.24 (m, 2H), 5.21 (d, J=1.0 Hz, 2H), 5.04 (d, J=12.3 Hz, 1H), 4.58 (d, J=12.3 Hz, 1H), 3.92 (s, 3H), 3.68 (d, J=1.7 Hz, 1H), 3.53 (s, 3H), 3.48 (s, 3H); MS (ESI) calculated for C₂₀H₂₃O₈ [M+H]⁺ 391.1, found 391.1.

### Example 4c: Preparation of (E)-1-(2-hydroxy-4-(methoxymethoxy)phenyl)-3-(4-methoxy-3-(methoxymethoxy)phenyl)prop-2-en-1-one (3D)

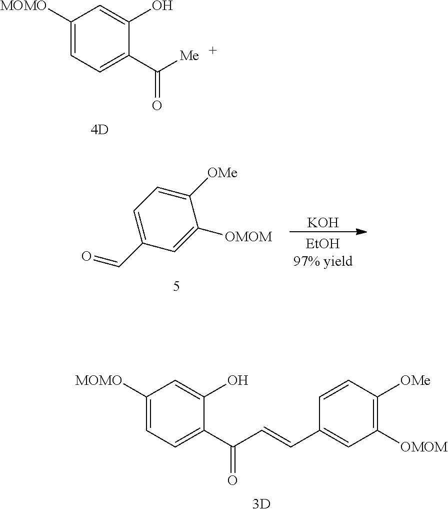

To a solution of 4D (example 4d, 740 mg, 3.8 mmol) in ethanol (20 mL) was added an aqueous KOH (50% wt, 5.4 mL, 75 mmol) followed by compound 5 (example 1e, 780 mg, 4.0 mmol) in EtOH (5 mL) was added dropwise. The reaction mixture was left stirred for 60 hours after which time LCMS analysis indicated consumption of the starting materials. The mixture was diluted with Et₂O (100 mL) and quenched with aqueous HCl solution (0.5N, 100 mL). The aqueous layers were extracted with Et₂O (3×100 mL). The ethereal layers were combined, dried over MgSO₄, filtered, and concentrated under reduced pressure. The crude residue was purified by silica gel chromatography (0→40% EtOAc/hexanes) to provide 0.8 g (57% yield) of chalcone 3D as a yellow solid. ¹H NMR (400 MHz, MeOD) δ 8.10 (d, J=9.0 Hz, 1H), 7.82 (d, J=15.3 Hz, 1H), 7.69 (d, J=15.3 Hz, 1H), 7.54 (d, J=2.1 Hz, 1H), 7.42 (dd, J=8.5, 2.2 Hz, 1H), 7.07 (d, J=8.5 Hz, 1H), 6.64 (dd, J=8.9, 2.5 Hz, 1H), 6.57 (d, J=2.5 Hz, 1H), 5.26 (d, J=2.1 H7, 4H), 3.90 (s, 3H), 3.53 (s, 3H), 3.47 (s, 4H); MS (ESI) calculated for C₂₀H²³O₇ [M+H]⁺ 375.1, found 375.0.

### Example 4d: Preparation of 1-(2-hydroxy-4,6-bis(methoxymethoxy)phenyl)ethan-1-one (4D)

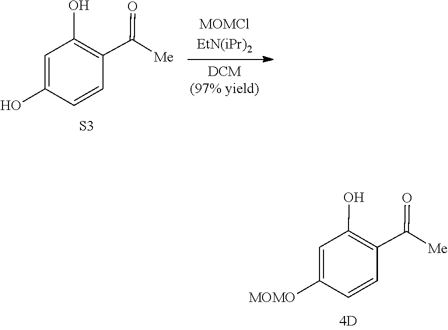

A 500 mL oven-dried round-bottom flask, equipped with stir bar and rubber septa, was charged with 2,4-dihydroxyacetophenone S3 (6.8 g, 45 mmol), followed by dry DCM (150 mL) under N₂. To the stirred suspension was slowly added DIPEA (14 mL, 80 mmol), resulting in a clear solution. To the stirred clear solution was added MOMCl (4.1 mL, 54 mmol) dropwise over 15 minutes. The reaction was left stirred for 18 hours, after which time LCMS analysis indicated consumption of the starting material. The solvent was removed under reduced pressure and the residue was purified by silica gel chromatography (0→40% EtOAc/hexanes) to provide 8.5 g (97% yield) of acetophenone 4D as a yellow oil. ¹H NMR (400 MHz, CDCl₃) δ 12.61 (s, 1H), 7.65 (d, J=8.8 Hz, 1H), 6.60 (d, J=2.4 Hz, 1H), 6.55 (dd, J=8.9, 2.5 Hz, 1H), 5.21 (s, 2H), 3.48 (s, 3H), 2.57 (s, 3H); MS (ESI) calculated for C₁₀H₁₃O₄ [M+H]⁺ 197.1, found 197.2.

### Example 5: Preparation of (2,3-trans)-5,7-dihydroxy-2-(3-hydroxy-4-methoxyphenyl)-4-oxochroman-3-yl propionate-methanol (105)

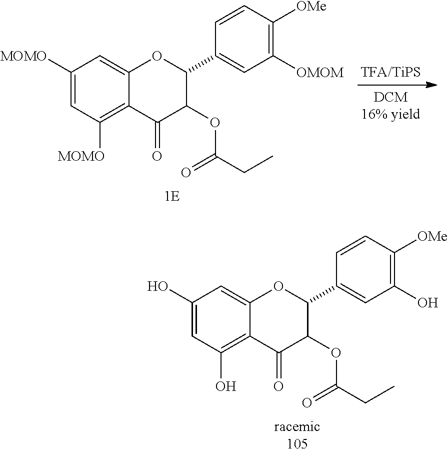

To a solution of 1E (example 5a, 15 mg, 30 μM) in DCM (2 mL) was added TiPS (10 μL) followed by TFA (1 mL). The resulting solution was left starred at 22° C. for 40 minutes, after which time LCMS analysis indicated consumption of the starting material. The solvent was removed under reduced pressure and the residue redissolved again in MeOH (5 mL) and the solvent was removed under reduced pressure. The crude residue was purified by preparative RP HPLC (10→90% MeCN/H₂O) to provide 1.8 mg (16% yield) of flavanonol 105 as a clear oil. ¹H NMR (500 MHz, MeOD) δ 7.05-6.85 (m, 3H), 6.03-5.87 (m, 2H), 5.80 (d, J=11.7 Hz, 1H), 5.29 (d, J=11.7 Hz, 1H), 3.89 (s, 3H), 2.35 (dq, J=16.7, 7.6 Hz, 1H), 2.32-2.15 (m, 1H), 1.02 (t, J=7.6 Hz, 3H); MS (ESI) calculated for C₁₉H₁₉O₆ [M+H]⁺ 375.1, found 375.0.

### Example 5a: Preparation of (2,3-trans)-2-(4-methoxy-3-(methoxymethoxy)phenyl)-5,7-bis(methoxymethoxy)-4-oxochroman-3-yl propionate (1E)

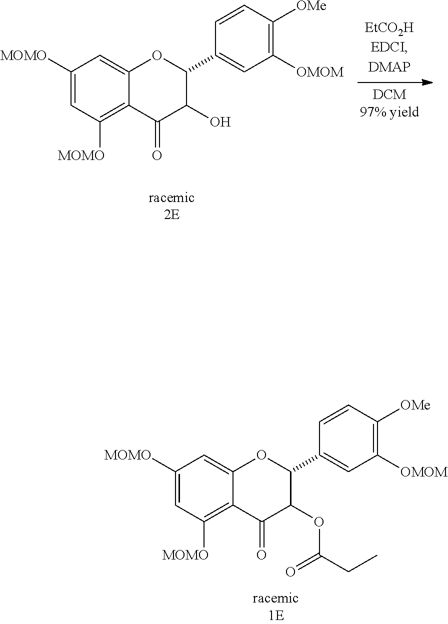

To a solution of 2E (example 5b, 14 mg, 31 μM) in DCM (3 mL) was added DMAP (7.6 mg, 62 μM), EDCl (12 mg, 62 μM) followed by a solution of propionic acid (4.6 mg, 62 μM) in DCM (1 mL). The resulting solution was left starred at 22° C. for 18 hours, after which time LCMS analysis indicated consumption of the starting material. The reaction mixture was directly loaded onto silica column and purified by silica gel chromatography (0 →50% EtOAc/hexanes) to provide 15 mg (97% yield) of ester S20 as a clear oil. ¹H NMR (500 MHz, CDCl₃) δ 7.05 (dd, J=8.4. 2.1 Hz, 1H), 6.85 (d, J=8.5 Hz, 1H), 6.41 (d, J=2.3 Hz, 1H), 6.30 (d, J=2.3 Hz, 1H), 5.61 (d, J=12.2 Hz, 1H), 5.28-5.14 (m, 5H), 5.14-4.96 (m, 2H), 3.83 (d, J=1.7 Hz, 3H), 3.45 (s, 6H), 3.40 (s, 3H), 2.31 (dq, J=16.6, 7.6 Hz, 1H), 2.26-2.07 (m, 1H), 0.98 (t, J=7.6 Hz, 3H); MS (ESI) calculated for C₂₅H₃₁O₁₁ [M+H]⁺ 507.5, found 507.2.

### Example 5b. Preparation of (trans)-3-hydroxy-2-(4-methoxy-3-(methoxymethoxy)phenyl)-5,7-bis(methoxymethoxy)chroman-4-one (2E)

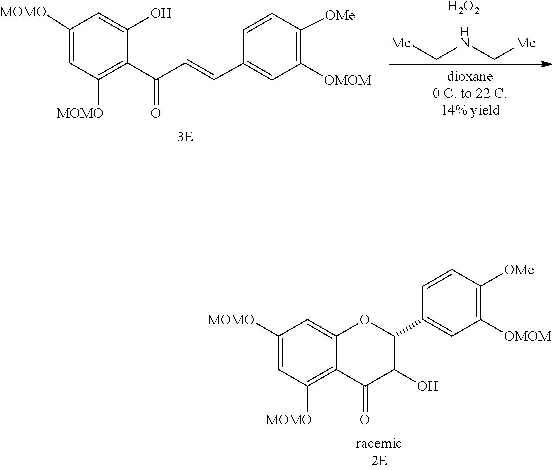

To an ice-cold (ice-bath) solution of 3E (example 5c, 3.77 g, 8.68 mmol) in dioxane (100 mL) was added Et₂NH (4.5 mL, 43 mmol) under N₂ followed by slow addition of H₂O₂ (30% wt); precipitation occurred upon addition of ~85 mL of H₂O₂ solution to afford 2E (557 mg, 14% yield) as a white solid. ¹H NMR (400 MHz, CDCl₃) δ 7.36 (d, J=2.1 Hz, 1H), 7.17 (dd, J=8.3, 2.1 Hz, 1H), 6.96 (d, J=8.4 Hz, 1H), 6.46 (d, J=2.2 Hz, 1H), 6.37 (d, J=2.2 Hz, 1H), 5.42-5.22 (m, 4H), 5.19-5.16 (m, 2H), 4.97 (d, J=12.2 Hz, 1H), 4.46 (d, J=12.2 Hz, 1H), 4.01 (s, 1H), 3.91 (s, 3H), 3.54 (s, 3H), 3.53 (s, 3H), 3.47 (s, 3H); MS (ESI) calculated for C₂₂H₂₇O₁₀ [M+H]⁺ 421.2, found 451.2.

### Example 5c: Preparation of (E)-1-(2-hydroxy-4,6-bis(methoxymethoxy)phenyl)-3-(4-methoxy-3-(methoxymethoxy)phenyl)prop-2-en-1-one (3E)

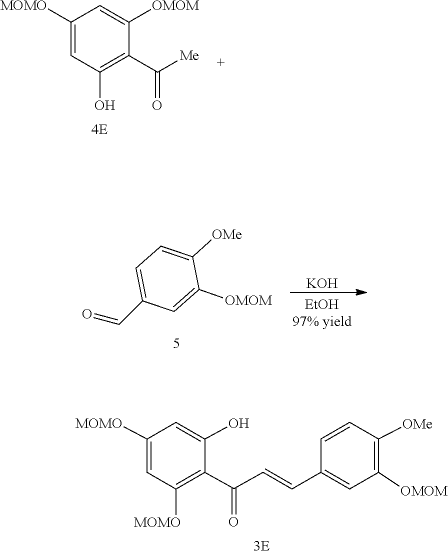

Prepare according to the procedure describe in example-1c from 4E (example 5d, 3.32 g, 13.0 mmol) in ethanol (60 mL), KOH (50% wt, 20 mL, 180 mmol), and 5 (example 1e, 2.67 g, 13.6 mmol) to give 3E (5.5 g, 97% yield) as a yellow oil. MS (ESI) calculated for C₂₂H₂₇O₉ [M+H]⁺ 435.2, found 435.2.

### Example 5d: Preparation of 1-(2-hydroxy-4,6-bis(methoxymethoxy)phenyl)ethan-1-one (4E)

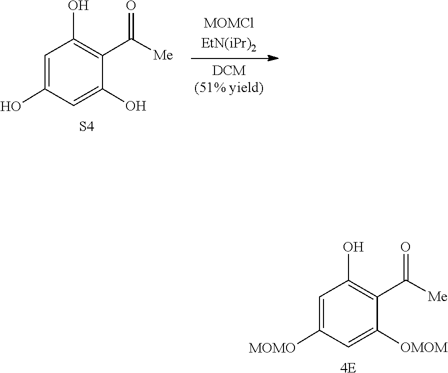

A 500 mL oven-dried round-bottom flask, equipped with stir bar and rubber septa, was charged with 2,4,6-trihydroxyacetophenone S4 (3.8 g, 23 mmol), followed by dry DCM (100 mL) under N₂. The mixture was cooled to 0° C. in an ice bath, and a DIPEA (11 mL, 63 mmol) was added slowly. After stirring for 20 min, MOMCl (3.8 mL, 50 mmol) was added dropwise to the suspension at 0° C. over 15 minutes. The reaction was left stirred for 4 hours (the ice bath was allowed to melt and suspension turned into light brown solution), after which time LCMS analysis indicated consumption of the starting materials. Water (100 mL) was added, followed by extraction with DCM (3×75 mL). The combined organic layers were dried over MgSO₄, filtered, and concentrated under reduced pressure. The crude residue was purified by silica gel chromatography (0→100% EtOAc/hexanes) to provide 3.0 g (51% yield) of acetophenone 4E as a clear oil, which solidified upon standing. ¹H NMR (400 MHz, DMSO-d₆) δ 13.34 (s, 1H), 6.23 (d, J=2.3 Hz, 1H), 6.19 (d, J=2.3 Hz, 1H), 5.30 (s, 2H), 5.23 (s, 2H), 3.44 (s, 3H), 3.38 (s, 3H), 2.60 (s, 3H); MS (ESI) calculated for C₁₂H₁₇O₆ [M+H]⁺257.1, found 257.2.

### Example 6: Preparation of (2,3-trans)-5-hydroxy-2-(4-hydroxyphenyl)-4-oxochroman-3-yl acetate (106)

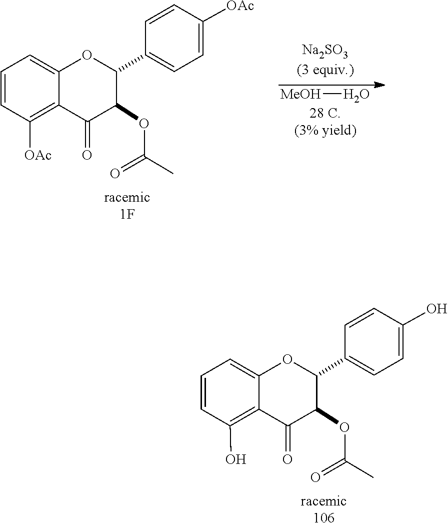

A 40 mL vial, equipped with stir bar and a Teflon cap, was charged with 1F (example 6a, 190 mg, 0.48 mmol), followed by MeOH (6 mL) and a solution of Na₂SO₃ (180 mg, 1.4 mmol) in H₂O (6 mL), under N₂. The resulting yellow solution was stirred at 28° C. for 18 hours and quenched with HCl (0.3 M, 6 mL) and extracted into EtOAc (3×25 mL), the combined organics were dried over MgSO₄, filtered and concentrated. Small amount was purified by recrystallization from MeCN—H₂O mixture to provide 4.2 mg (3% yield) of 106 as a white solid. ¹H NMR (400 MHz, CDCl₃) δ 11.18 (s, 1H), 7.42 (t, J=8.3 Hz, 1H), 7.39-7.32 (m, 2H), 6.95-6.81 (m, 2H), 6.59 (dd, J=8.4, 0.9 Hz, 1H), 6.50 (dd, J=8.2, 1.0 Hz, 1H), 5.84 (d, J=12.1 Hz, 1H), 5.34 (d, J=12.1 Hz, 1H), 4.84 (s, 1H), 2.04 (s, 3H); MS (ESI) calculated for C₁₅H₁₁O₄ [M−OAc]⁺ 255.1, found 255.2.

### Example 6a: Preparation of (2,3-trans)-2-(4-acetoxyphenyl)-4-oxochromane-3,5-diyl diacetate (1F)

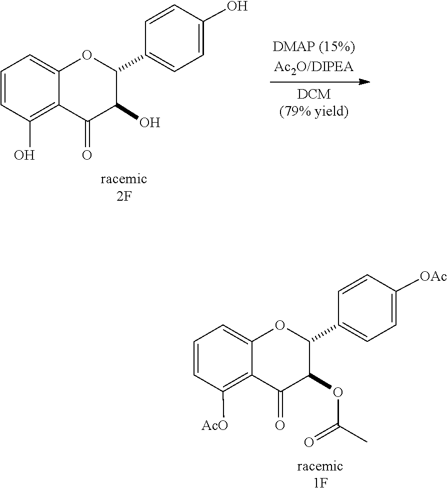

A 200 mL round-bottom vial, equipped with stir bar and rubber septa, was charged with 1F (example 6b, 234 mg, 745 μmol, as a 3:1 mixture of trans-cis isomers), followed by dry DCM (40 mL). To the mixture was added acetic anhydride (380 μL, 4.1 mmol) followed by DIPEA (860 μL, 5.2 mmol). After stirring for 5 minutes, a solution of DMAP (14 mg, 110 μmol) in DCM (3 mL) was added. The reaction was left stirred for 21 hours, after which time LCMS analysis indicated consumption of the starting materials. Water (40 mL) was added, followed by extraction with DCM (2×40 mL). The combined organic layers were dried over MgSO₄, filtered, and concentrated under reduced pressure. preparative RP HPLC (10→90% MeCN/H₂O) to provide 233 mg (79% yield, 10:1 mixture trans: cis) of flavanonol triacetate 2F as a white solid. Small amount of the trans-cis mixture was purified to pure trans-isomer by trituration with MeOH. ¹H NMR (400 MHz, CDCl₃) δ 7.57-7.44 (m, 3H), 7.22-7.10 (m, 2H), 6.96 (dd, J=8.5, 1.1 Hz, 1H), 6.76 (dd, J=8.0, 1.0 Hz, 1H), 5.73 (d, J=12.4 Hz, 1H), 5.44 (d, J=12.3 Hz, 1H), 2.39 (s, 3H), 2.32 (s, 3H), 2.02 (s, 3H).; MS (ESI) calculated for C₂₁H₁₉O₈ [M+H]⁺ 399.0, found 399.0.

### Example 6b. Preparation of (2,3-trans)-3,7-dihydroxy-2-(4-hydroxyphenyl) chroman-4-one-1-methanol (1/1) (2E)

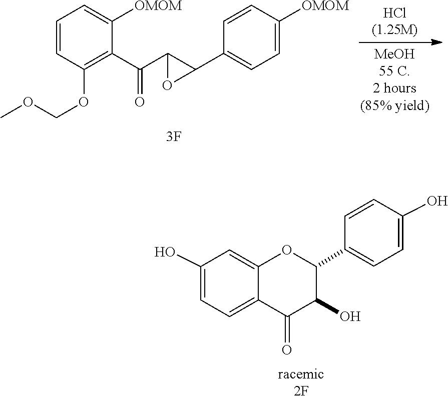

A 40 mL round-bottom vial, equipped with stir bar and Teflon cap was charged with 3F (example 6c, 410 mg, 1.0 mmol), followed by dry methanolic HCl (1.25 M, 12 mL) under N₂. The reaction mixture was heated at 55° C. for 2 hours, after which time LCMS analysis indicated consumption of the starting material. Solvent was removed under reduced pressure and the residue was redissolved in MeOH (20 mL) and the solvent was removed under reduced pressure. The crude residue was purified by preparative RP HPLC (10→90% MeCN/H₂O) to provide 234 mg (85% yield) of flavanonol 2F as a white solid (~3:1 mixture of trans and cis isomers, epimerization may occur during purification operations). Small amount of the trans-cis mixture was purified to pure trans-isomer by trituration with MeOH. ¹H NMR (400 MHz, MeOD) δ 7.42 (t, J=8.3 Hz, 1H), 7.39-7.32 (m, 2H), 6.91-6.80 (m, 2H), 6.51 (d, J=8.3 Hz, 1H), 6.46 (d, J=8.3 Hz, 1H), 5.07 (d, J=11.9 Hz, 1H), 4.66 (d, J=11.9 Hz, 1H); MS (ESI) calculated for C₁₅H₁₃O₅ [M+H]⁺ 273.1, found 273.2.

### Example 6c: Preparation of (E)-1-(2-hydroxy-4,6-bis(methoxymethoxy)phenyl)-3-(4-methoxy-3-(methoxymethoxy)phenyl)prop-2-en-1-one (3E)

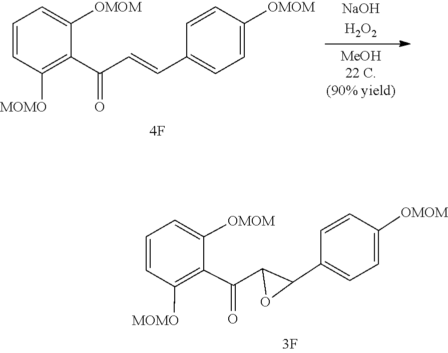

To a solution of chalcone 4F (example 6d, 1.34 g, 3.45 mmol) in methanol (100 mL) was added NaOH (0.4 g, 10 mmol) at 22° C. in one portion followed by a dropwise addition of aqueous solution of hydrogen peroxide (30% wt, 2.8 mL, 28 mmol). The reaction mixture was left stirred for 24 hours, after which time LCMS analysis indicated consumption of the starting material. The reaction was diluted with Et₂O (75 mL), washed with a saturated aqueous NH₄Cl solution (2×75 mL) and extracted with Et₂O (2×75 mL). The organics were combined, dried over MgSO₄, filtered and concentrated under reduced pressure to provide 1.26 g (90% yield) of chalcone epoxide 3F as a light yellow oil. ¹H NMR (400 MHz, CDCl₃) δ 7.29 (d, J=8.4 Hz, 1H), 7.25-7.17 (m, 2H), 7.07-6.96 (m, 2H), 6.82 (d, J=8.4 Hz, 2H), 5.17 (s, 2H), 5.15 (s, 2H), 5.15 (s, 2H), 3.99 (d, J=1.8 Hz, 1H), 3.89 (d, J=1.9 Hz, 1H), 3.46 (s, 3H), 3.42 (s, 6H); MS (ESI) calculated for C₂₁H₂₅O₈ [M+H]⁺ 405.2, found 405.0.

### Example 6d: Preparation of (E)-1-(2,6-bis(methoxymethoxy)phenyl)-3-(4-(methoxymethoxy)phenyl)prop-2-en-1-one (4F)

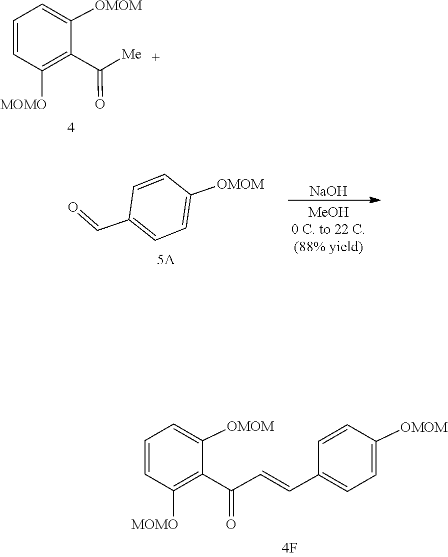

To a solution of 4 (example 1d, 0.94 g, 3.9 mmol) in methanol (10 mL) was added an aqueous NaOH (30% wt, 5 mL, 35 mmol) at 22° C. followed by a solution of 5A (example 6e, 0.72 g, 4.3 mmol) in MeOH (7 mL). The reaction mixture was left stirred for 18 hours, after which time LCMS analysis indicated consumption of the starting materials. The mixture was diluted with Et₂O (50 mL) and washed with saturated aqueous NH₄Cl solution (2×25 mL). The aqueous layers were combined and extracted with (2×50 mL Et₂O). The organic layers were combined, dried over MgSO₄, filtered, and concentrated under reduced pressure. The crude residue was purified by silica gel chromatography (0→100% EtOAc/hexanes) to provide 1.34 g (88% yield) of chalcone 4F as a light yellow oil. ¹H NMR (400 MHz, CDCl₃) δ 7.54-7.42 (m, 2H), 7.34-7.24 (m, 2H, overlap with CHCl₃), 7.09-6.96 (m, 2H), 6.95-6.77 (m, 3H), 5.19 (s, 2H), 5.13 (s, 4H), 3.47 (s, 3H), 3.38 (s, 6H); MS (ESI) calculated for C₂₁H₂₅O₇ [M+H]⁺ 389.2, found 389.2.

### Example 6e: Preparation of 4-(methoxymethoxy)benzaldehyde (5A)

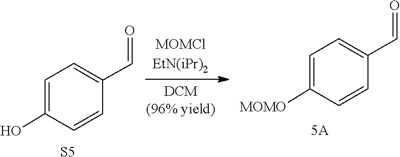

A 1 L oven-dried round-bottom flask, equipped with stir bar and rubber septa, was charged with 4-hydroxybenzaldehyde S5 (12 g, 98 mmol), followed by dry DCM (250 mL) under N₂. The mixture was cooled to 0° C. in an ice bath, and DIPEA (24 mL, 140 mmol) was added followed by MOMCl (8.2 mL, 110 mmol) was added dropwise to the suspension at 0° C. over 15 minutes. The reaction was left stirred for 18 hours (the ice bath was allowed to melt), after which time LCMS analysis indicated consumption of the starting materials. Water (100 mL) was added, followed by extraction with DCM (3×75 mL). The combined organic layers were dried over MgSO₄, filtered, and concentrated under reduced pressure. The crude residue was purified by silica gel chromatography (0→50% EtOAc/hexanes) to provide 15.7 g (96% yield) of aldehyde 5A as clear oil. ¹H NMR (400 MHz, CDCl₃) δ 9.90 (s, 1H), 7.92-7.74 (m, 2H), 7.22-7.05 (m, 2H), 5.25 (s, 2H), 3.49 (s, 3H); MS (ESI) calculated for C₉H₁₁O₃ [M+H]⁺ 167.1, found 167.0.

### Example 7—Compound Testing

Each of Compounds 101-106 was synthesized. Thereafter, the compounds were tested in an in vitro cell-based assay having cells that overexpress the TIR sweet taste receptor. The sweet dose-response curves were recorded and the EC50 was determined. Table 2 summarizes calculated EC50 values for certain tested compounds.

TABLE 2

| Compound | EC50 (μM) |
|---|---|
| 101 | ~1 |
| 102 | ~2 (partial agonist) |
| 103 | ~8 |
| 104 | ~10 |
| 105 | ~5 |
| 106 | ~45 |

### Example 8—Sensory Testing: Sweetness Alone

The test samples were prepared from Compound 101 (4 ppm aqueous solution) and Compound 103 (7 ppm aqueous solution). Each of the test samples was evaluated by a sensory panel for its sweetness relative to 1.5 wt % sucrose solution. The panelists were asked to choose the sweeter sample in a pair of a compound test sample and a 1.5 wt % solution of sucrose. For Compound 101, the taste sample was selected 14 times as sweeter, while the sucrose control was selected 46 times out of total 60 times. For Compound 103, the taste sample was selected 14 times as sweeter, while the sucrose control was selected 55 times out of total 69 times.

### Example 9—Sensory Testing: Enhancement

Two control samples were prepared of 6 wt % sucrose and 7.5 wt % sucrose in Low sodium buffer. Two test samples were prepared: one at 6 wt % sucrose plus 4 ppm of Compound 101, and the other at 6 wt % sucrose plus 7 ppm of Compound 103. Panelists were asked to evaluate the four samples and rate the sweetness on a scale of 0 to 10, with a higher rating indicating greater sweetness. Table 3 shows the mean score by panelists

TABLE 3

| Sample | Sweetness Score |
|---|---|
| 6 wt % sucrose | 6.6 |
| 7.5 wt % sucrose | 8.6 |
| 6 wt % sucrose + 4 ppm Compound 101 | 9.8 |
| 6 wt % sucrose + 7 ppm Compound 103 | 8.8 |

1. A flavor-modifying compound, which is compound of formula (I): 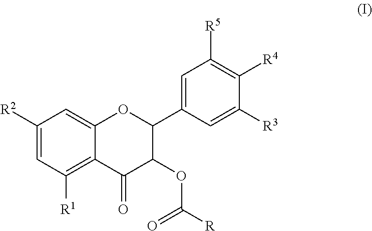
or a salt thereof, wherein:
R¹ and R² are, independently of each other, a hydrogen atom, —OH, or —OCH₃;
R³, R⁴, and R⁵ are, independently of each other, a hydrogen atom, —OH, or —OCH₃; and
R is methyl, ethyl, isopropyl, or benzyl.

2. The flavor-modifying compound of claim 1, wherein both of R¹ and R² are —OH.

3. The flavor-modifying compound of claim 1, wherein one of R¹ and R² is a hydrogen atom, and the other is a hydrogen atom.

4. The flavor-modifying compound of claim 1, wherein R⁴ is —OH and R³ and R⁵ are each a hydrogen atom.

5. The flavor-modifying compound of claim 1, wherein R³ is —OH, R⁴ is
—OCH₃, and R⁵ is a hydrogen atom.

6. The flavor-modifying compound of claim 1, wherein R is methyl or ethyl.

7. The flavor-modifying compound of claim 1, which is 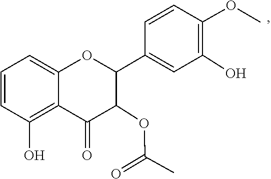 [SMILES: COc1ccc(C2Oc3cccc(O)c3C(=O)C2OC(C)=O)cc1O] 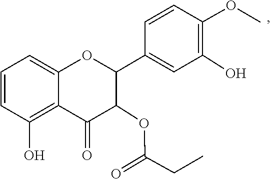 [SMILES: CCC(=O)OC1C(=O)c2c(O)cccc2OC1c1ccc(OC)c(O)c1] 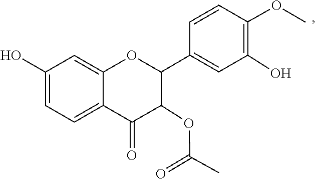 [SMILES: COc1ccc(C2Oc3cc(O)ccc3C(=O)C2OC(C)=O)cc1O] 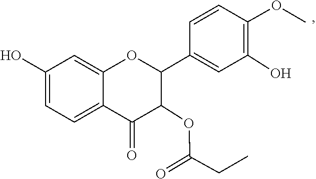 [SMILES: CCC(=O)OC1C(=O)c2ccc(O)cc2OC1c1ccc(OC)c(O)c1] 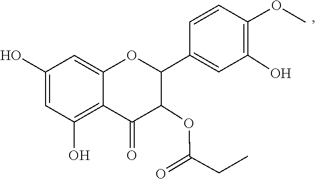 [SMILES: CCC(=O)OC1C(=O)c2c(O)cc(O)cc2OC1c1ccc(OC)c(O)c1] 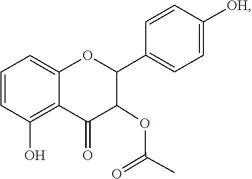 [SMILES: CC(=O)OC1C(=O)c2c(O)cccc2OC1c1ccc(O)cc1]
or a comestibly acceptable salt of any of the foregoing.

8. Use of a flavor-modifying compound of claim 1 to reduce a bitter taste of an ingestible composition.

9. The use of claim 8, wherein the concentration of the flavor-modifying compound used in the ingestible composition ranges from 0.1 ppm to 1000 ppm.

10. The use of claim 8, wherein the ingestible composition comprises one or more bitter tastants.

11. The use of claim 10, wherein the bitter tastant is a high-intensity sweetener, selected from the group consisting of: acesulfame potassium, aspartame, neotame, cyclamate, saccharin, sucralose, rebaudioside A, rebaudioside B, rebaudioside M, rebaudioside D, rebaudioside E, mogroside III, mogroside IV, mogroside V, siamenoside I, isomogroside V, mogroside IV_{E}, isomogroside IV, mogroside III_{E}, 11-oxomogroside V, the 1,6-α isomer of siamenoside I, or any combinations thereof.

12. The use of claim 10, wherein the bitter tastant is potassium chloride.

13. The use of claim 10, wherein the bitter tastant is selected from the group consisting of: pea protein, soy protein, almond protein, cashew protein, canola protein, chickpea protein, fava protein, sunflower protein, wheat protein, oat protein, barley protein, potato protein, or any combinations thereof.

14. The use of claim 10, wherein the bitter tastant is selected from the group consisting of: atropine, brinzolamide, chloramphenicol, chloroquine, clindamycin, dexamethasone, digoxin, diltiazem, diphenhydramine, docusate, dorzolamide, doxepin, doxylamine, enalapril, erythromycin, esomeprazole, famotidine, gabapentin, ginkgolide A, guaifenesin, L-histidine, lomefloxacin, methylprednisolone, ofloxacin, oleuropein, oxyphenonium, pirenzepine, prednisone, ranitidine, trapidil, trimethoprim, cetirizine, or any combinations thereof.

15. Use of a flavor-modifying compound of claim 1 to enhance a sweet taste of an ingestible composition.

16. The use of claim 15, wherein the concentration of the flavor-modifying compound used in the ingestible composition ranges from 0.1 ppm to 1000 ppm.

17. The use of claim 15, wherein the ingestible composition comprises one or more bitter tastants.

18. An ingestible composition, which comprises a flavor-modifying compound of claim 1 and a bulking agent.

19. The ingestible composition of claim 18, wherein the ingestible composition comprises a sweetener.

20. The ingestible composition of claim 18, wherein the flavor-modifying compound is present in the ingestible composition at a concentration ranging from 0.1 ppm to 1000 ppm.

21. A flavored product, which comprises the ingestible composition of claim 18.

22. The flavored product of claim 21, which is a beverage product, a food product, an oral care product, or a pharmaceutical product.
